(19) United States
(12) Patent Application Publication
(10) Pub. No.: US 2026/0134982 A1
(43) Pub. Date: May 14, 2026

(54) Managing Ultrasound Devices

(71) Applicant: FUJIFILM SonoSite, Inc., Bothell, WA (US)

(72) Inventors: Craig Chamberlain, Seattle, WA (US); Saeed Aliakbari, Snohomish, WA (US); Katsuya Yamamoto, Kaisei-machi (JP); Christopher Howard, Seattle, WA (US); Thomas J. Endres, Sagle, ID (US); Mark Fiebig, Snohomish, WA (US)

(73) Assignee: FUJIFILM SonoSite, Inc., Bothell, WA (US)

(21) Appl. No.: 18/947,838

(22) Filed: Nov. 14, 2024

(51) Int. Cl. G16H 40/40; A61B 8/00

(52) U.S. Cl. CPC G16H 40/40; A61B 8/4263; A61B 8/4405; A61B 8/4422; A61B 8/465; A61B 8/56; A61B 8/58

(57) ABSTRACT

Systems and methods for managing ultrasound devices are disclosed. Multiple ultrasound devices, such as ultrasound scanners, ultrasound machines, ultrasound carts, etc., can be tracked. For example, the ultrasound device's location (e.g., geotag location) or network connection can be tracked and used to determine a state of the ultrasound device on a state machine. The system can then initiate one or more actions appropriate to the state of the ultrasound device. Additionally, the system includes containers for charging, cleaning, and testing ultrasound devices. The system can also include a charger array on a table, wall, or cart to wirelessly charge the ultrasound device. The ultrasound device can include an inertial measurement unit (IMU), which can enable a user to move the device in the air to perform a gesture with the device, and resulting IMU data can be used to trigger an action by the system.

### BACKGROUND

Ultrasound devices, particularly ultraportable devices such as wireless scanners (e.g., wireless probes), have monetary value and can be easy to conceal due to their small form factor. Hence, and unfortunately, ultrasound devices are frequently stolen from care facilities. In other cases, ultrasound devices are misplaced and ultimately lost. Equipment administrators are typically unsure if a missing ultrasound device is lost or stolen and, thus, are unsure if or when to replace the device. Therefore, the ultrasound devices may not be available when needed for patient care, resulting in a patient not receiving the best care possible.

Point-of-care (POC) ultrasound machines are often placed on a mobile stand and can be electronically connected to the stand (e.g., as a power source) by cables. The cables are cumbersome to use, can collect dirt and debris, and can present a hazard during use of the POC ultrasound machine. Moving the ultrasound machine from the stand to a desktop configuration generally requires disconnecting and reconnecting power cables, which is cumbersome and time-consuming. Electrical connectors of the ultrasound machine used for charging are fragile and often fail. Wireless ultrasound devices (e.g., scanners) are typically cleaned, disinfected, and charged sequentially, which requires additional time. These deficiencies can prevent an ultrasound system from being ready for use, resulting in patients not receiving the best care possible.

### SUMMARY

Systems and methods for managing ultrasound devices are disclosed. Multiple ultrasound devices, such as ultrasound scanners, ultrasound machines, ultrasound carts, etc., can be tracked. For example, the ultrasound device's location (e.g., geotag location) or network connection can be tracked and used to determine a state of the ultrasound device on a state machine. The system can then initiate one or more actions appropriate to the state of the ultrasound device. Additionally, the system includes containers for charging, cleaning, and testing ultrasound devices. The system can also include a charger array on a table, wall, or cart to wirelessly charge the ultrasound device. The ultrasound device can include an inertial measurement unit (IMU), which can enable a user to move the device in the air to perform a gesture with the device, and the resulting IMU data can be used to trigger an action by the system.

In some aspects, an ultrasound system is disclosed. The ultrasound system includes a state machine configured to manage a state of each of a plurality of ultrasound devices connected to a network, the state machine configured to determine a combination of conditions met by an ultrasound device of the plurality of ultrasound devices, determine a state of the ultrasound device based on the combination of conditions, different combinations of conditions each being associated with a different state of a plurality of states, and initiate one or more actions based on the state of the ultrasound device.

In some aspects, a system is disclosed. The system includes a container having a drawer for storing ultrasound devices. The drawer includes a mount configured to couple to an ultrasound device, a charger transmitter configured to provide wireless charging signals to the ultrasound device, one or more cleaning sources configured to emit a light to clean and disinfect the ultrasound device, and a fluid source configured to emit a fluid into the drawer to clean and disinfect the ultrasound device.

In some aspects, a method for managing ultrasound devices is disclosed. The method includes determining, by a state machine of an ultrasound system, a combination of conditions met by an ultrasound device of a plurality of ultrasound devices communicatively coupled to the ultrasound system via a network. The method also includes selecting a first state from a plurality of states of the state machine based on the combination of conditions met by the ultrasound device, each state of the plurality of states corresponding to a different combination of conditions. Additionally, the method includes transitioning a state of the ultrasound device to the first state and, responsive to transitioning the ultrasound device to the first state, initiating one or more actions for the ultrasound device.

In some aspects, a method for managing ultrasound devices is disclosed. The method includes determining that an ultrasound device is mounted on and communicatively coupled to a mount disposed within a drawer of a container that is usable to simultaneously charge, clean, and test ultrasound devices. The method also includes providing wireless charging signals, via one or more charger transmitters disposed within the drawer, to the ultrasound device to charge a battery of the ultrasound device located within the drawer. In addition, the method includes providing light from a cleaning source or fluid from a fluid source onto the ultrasound device to clean and disinfect the ultrasound device located within the drawer. The method further includes running a calibration routine on the ultrasound device located within the drawer to check for faults and adjust parameters according to data acquired during the calibration routine. Further, the method includes providing data corresponding to the ultrasound device located within the drawer via a display disposed on an exterior surface of the drawer, the data including a state of the ultrasound device in a state machine. In some aspects, the method can further include detecting a temperature of the ultrasound device located within the drawer. The method also includes determining whether the temperature of the ultrasound device is below a temperature threshold. If the temperature of the ultrasound device is above the temperature threshold, then the drawer remains locked and the method continues monitoring the temperature of the ultrasound device until the temperature cools to a safe temperature. If the temperature of the ultrasound device is at or below the temperature threshold, then the drawer is automatically unlocked to enable access to the ultrasound device such that a user can remove the ultrasound device from the drawer.

### BRIEF DESCRIPTION OF THE DRAWINGS

The appended drawings illustrate examples and are, therefore, exemplary embodiments and not considered to be limiting in scope. Throughout the drawings, the same numbers are used to reference like features and components.

FIG. 1 illustrates an ultrasound system in an environment for managing ultrasound devices in accordance with the some of the disclosed implementations.

FIG. 2 illustrates an example implementation of the ultrasound system illustrated in the environment of FIG. 1.

FIG. 3 illustrates an example state machine for managing ultrasound devices in accordance with some of the disclosed implementations.

FIG. 4 illustrates an example system for managing ultrasound devices, including charging of ultrasound devices.

FIG. 5 illustrates an example system for managing ultrasound devices, including charging of ultrasound devices.

FIG. 6 illustrates an example system for managing ultrasound devices, including charging of ultrasound devices.

FIG. 7 illustrates an example system for managing ultrasound devices, including charging, cleaning, and testing of ultrasound devices.

FIG. 8 illustrates an example environment for managing ultrasound devices, including an ultrasound scanner having a lightbar, and an ultrasound system.

FIG. 9 illustrates a block diagram of an example charger device having an integrated cooler.

FIG. 10 illustrates a block diagram of an example computing device that can perform one or more of the operations described herein, in accordance with some implementations.

FIG. 11 illustrates an example user interface for managing ultrasound devices.

FIG. 12 depicts a method for managing ultrasound devices, in accordance with one or more implementations.

FIG. 13 depicts a method for managing ultrasound devices, in accordance with one or more implementations.

FIG. 14 depicts a method for managing ultrasound devices, in accordance with one or more implementations.

### DETAILED DESCRIPTION

Many ultrasound devices, such as portable and ultraportable devices (e.g., wireless scanners), often go missing, and equipment administrators are typically unsure if the missing device is lost or stolen and, thus, unsure if or when to replace the device. In addition, many point-of-care (POC) ultrasound machines, although portable, include cumbersome power cables that need to be disconnected and reconnected frequently when switching from a mobile stand to a desktop configuration. Further, ultrasound devices are typically cleaned, disinfected, and charged sequentially, which requires additional time. These deficiencies can prevent an ultrasound system from being ready for use, resulting in patients not receiving the best care possible.

Disclosed herein are systems and methods for managing ultrasound devices. The systems and methods include a variety of features that enable tracking of ultrasound devices, charging, cleaning, and testing the ultrasound devices, and determining whether an ultrasound device is ready for use or not. The system includes a state machine that tracks a state of ultrasound devices coupled to a network. Based on a combination of conditions met by an ultrasound device, the state machine determines the state of the ultrasound device. Upon transitioning the ultrasound device to the determined state, the system can trigger or initiate specific actions corresponding to that state. For example, if the ultrasound device is determined to be lost or stolen, the system can implement actions to attempt to recover the missing ultrasound device. In another example, if the ultrasound device needs to be serviced, the system can initiate actions to have the ultrasound device sent to a service department for repairs.

The systems and methods can also be implemented to provide non-contact charging to the ultrasound device. For example, a charger array can be implemented on a wall to provide wireless charging signals to an ultrasound cart placed in proximity of the charging signals. An ultrasound machine mounted on the ultrasound cart can also be charged, by either the charger array on the wall, the ultrasound cart, or both. In another example, a charger array can be implemented on, in, or under a table (e.g., a desktop) such that the ultrasound machine can be charged when it is placed on the table. If the table is an examination table and the ultrasound device is a wireless ultrasound scanner, the scanner can be charged and geolocated while it is being used to scan a patient lying on the examination table.

The systems and methods can also be implemented to simultaneously charge, clean, and test an ultrasound device. For example, a container with drawers can include one or more charger transmitters, cleaning sources, fluid sources, calibration tools, etc. for charging, cleaning, disinfecting, and testing an ultrasound device located within a drawer. Further, a display can be included on the exterior of the drawer to provide information corresponding to the ultrasound device located within the drawer. Thermal sensors can be included on the ultrasound device or in the drawer to detect and monitor the temperature of the ultrasound device. The temperature can be used by the system to determine when it is safe (e.g., when the ultrasound device has cooled to below a threshold temperature) for a clinician to touch the ultrasound device after the ultrasound device has been cleaned and charged, due to the elevated temperatures experienced by the ultrasound device during a maintenance cycle.

The systems and methods can also include a user interface for managing ultrasound devices. The user interface can be displayed on any suitable computing device and enables a user to select options from various user-selectable options to manage the ultrasound devices on the network. Selecting an option in one panel can cause another panel to display additional information associated with the selected option. For example, a user can select a missing state in order to view information corresponding to one or more ultrasound devices that are missing. Each panel can include additional user-selectable options that are selectable to obtain more detailed information in another panel. These and other features are described in further detail below.

### Example Ultrasound System

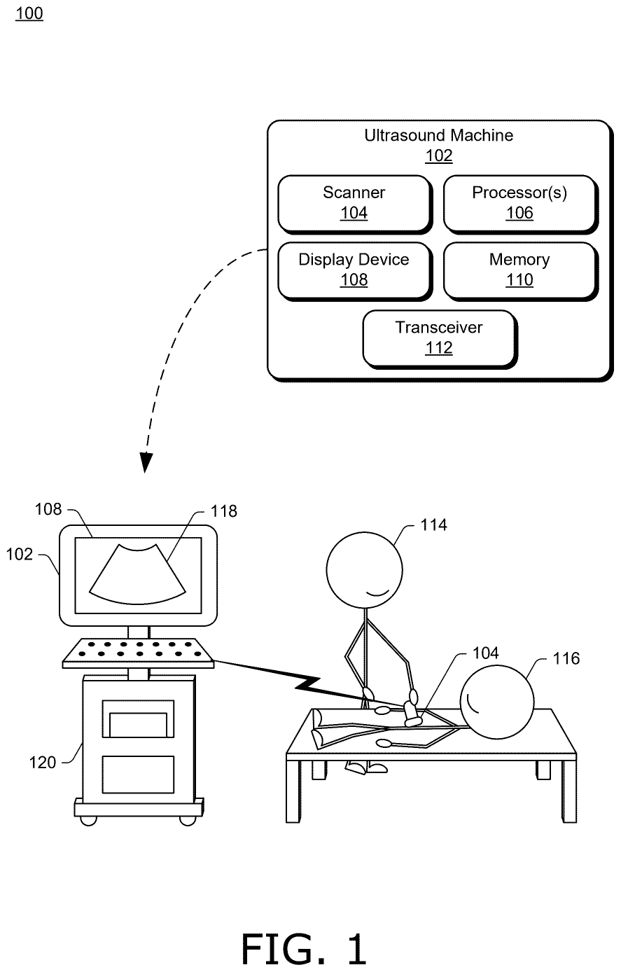

FIG. 1 illustrates an ultrasound system 100 in an environment for managing ultrasound devices in accordance with the disclosed implementations. The ultrasound system 100 in FIG. 1 includes an ultrasound machine 102 and an ultrasound scanner (e.g., scanner 104). The ultrasound machine 102 generates high-frequency sound waves (e.g., ultrasound) and imaging data based on the ultrasound reflecting off a patient anatomy/body structure and/or an interventional instrument (e.g., a needle). The ultrasound machine 102 includes various components, some of which include the scanner 104, one or more processors 106, a display device 108, a memory 110, and a transceiver 112.

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116, an interventional instrument, etc., for testing, diagnostic, therapeutic, or procedural reasons. In some implementations, the scanner 104 includes an ultrasound transducer array and electronics communicatively coupled to the ultrasound transducer array to transmit ultrasound signals to the patient's anatomy and receive ultrasound signals reflected from the patient's anatomy. In some implementations, the scanner 104 can also be referred to as an ultrasound probe or transducer. In embodiments, the scanner 104 is a multi-array scanner. For instance, a multi-array scanner in accordance with the present disclosure can include one or more of the arrays described in U.S. patent application Ser. No. 18/613,694 filed on Mar. 22, 2024, and entitled Multi-Dimensional and Multi-Frequency Ultrasound Transducers to Zhang et al., the disclosure of which is incorporated herein by reference in its entirety. A multi-array scanner in accordance with the present disclosure can include one or more of the arrays described in U.S. patent application Ser. No. 17/561,313 filed on Dec. 23, 2021, and entitled Array Architecture and Interconnection for Transducers to Li et al., the disclosure of which is incorporated herein by reference in its entirety. Because multi-array scanners can be more versatile and more valuable in terms of monetary cost than some other scanners, the state machine can treat the multi-array scanners differently from other scanners or device by, for example, assigning a higher priority level to a multi-array scanner, using a shorter count-down timer to declare the scanner missing, scheduling a replacement without waiting for the state of the multi-array scanner to advance to a stolen state, or prioritize the multi-array scanner over other devices when charging multiple devices simultaneously.

The display device 108 is coupled to the processor(s) 106, which can include any suitable processor, number of processors, or processor system, such as one or more central processing units (CPUs), graphics processing units (GPUs), vector processors, reduced instruction set computer (RISC) processors, complex instruction set computer (CISC) processors, very long instruction word (VLIW) processors, etc. The processor 106 can execute instructions stored on the memory 110 to perform operations disclosed herein for managing ultrasound devices. For example, the processor 106 can process the reflected ultrasound signals to generate ultrasound data, including an ultrasound image (e.g., ultrasound image 118). The display device 108 is configured to generate and display an ultrasound image (e.g., ultrasound image 118) of the anatomy and/or interventional instrument (e.g., a port) based on the ultrasound data generated by the processor 106 from the reflected ultrasound signals detected by the scanner 104. In some aspects, the ultrasound data includes the ultrasound image 118 or data representing the ultrasound image 118. The transceiver 112 can be configured to transmit (e.g., over a network maintained by a care facility) the ultrasound data and/or any data related to the ultrasound examination, such as medical worksheet data, etc., to a medical archiver (e.g., a vendor neutral archive (VNA)). In embodiments, the transceiver 112 can receive data from the medical archiver, such as patient history data or previous examination data. In some implementations, the ultrasound machine 102 can be mounted on, placed on, rested on, and/or connected to an ultrasound cart 120, which can provide various support features and functions for the ultrasound machine 102, including power, input devices, transportability, mounting support, etc.

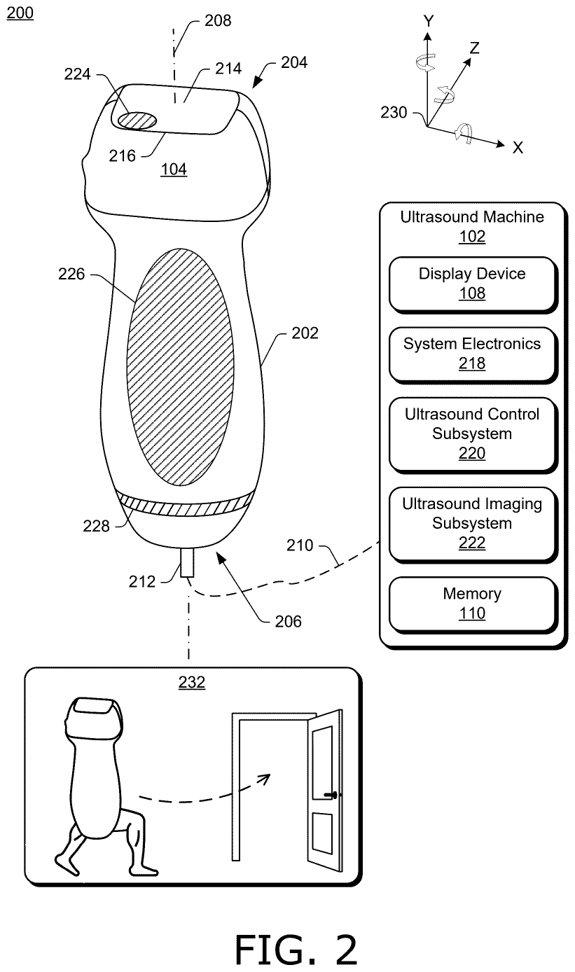

FIG. 2 illustrates an example implementation 200 of the ultrasound system 100 illustrated in FIG. 1. In the implementation 200, the scanner 104 (e.g., ultrasound scanner) can be any suitable type of ultrasound scanner. In an example, the scanner 104 is configured for handheld operation (e.g., external to a patient's body). The scanner 104 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. The scanner 104 is electrically coupled to an ultrasound imaging system (e.g., the ultrasound machine 102) via a coupling 210. In one example, the coupling 210 includes a cable that is attached to the proximal end portion 206 of the scanner 104 by a strain-relief element 212. In some implementations, though, the coupling 210 includes a wireless coupling so that the scanner 104 is wirelessly coupled to the ultrasound imaging system and communicates with the ultrasound imaging system via one or more wireless transmitters, receivers, or transceivers over a wireless connection or network (Bluetooth™, Wi-Fi™, etc.).

The scanner 104 includes a transducer assembly 214 at the distal end portion 204 of the scanner 104. The transducer assembly 214 is disposed under an acoustic lens (e.g., lens 216) of the scanner 104. The transducer assembly 214 can have one or more transducer elements electrically coupled to system electronics 218 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 218 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

In some implementations, the system electronics 218 include one or more processors (e.g., the processor(s) 106 from FIG. 1), integrated circuits, application-specific integrated circuits (ASICs), field programmable gate arrays (FPGAs), and power sources to support functioning of the ultrasound machine 102. In some implementations, the ultrasound machine 102 also includes an ultrasound control subsystem 220 having one or more processors. At least one processor, FPGA, or ASIC can cause electrical signals to be transmitted to the transducer(s) of the scanner 104 to emit sound waves and also receive electrical pulses from the transducer(s) that were created from the returning echoes. One or more processors, FPGAs, or ASICs can process the raw data associated with the received electrical pulses and form an image that is sent to an ultrasound imaging subsystem 222, which causes the image (e.g., the image 118 in FIG. 1) to be displayed via the display device 108. Thus, the display device 108 displays ultrasound images from the ultrasound data processed by the processor(s) of the ultrasound control subsystem 220.

In some implementations, the ultrasound machine 102 also includes one or more user input devices (e.g., a keyboard, a cursor control device, a microphone, a camera, a touchscreen, etc.) that input data and enable obtaining measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In aspects, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the acquired data. To avoid obscuring the techniques described herein, such user input devices, disk storage device, and printer are not shown in FIG. 2.

The scanner 104 in the implementation 200 also includes one or more pressure sensors 224 on the lens 216 of the scanner 104, and one or more pressure sensors 226 on the enclosure 202 of the scanner 104. The pressure sensors 224 and 226 can include in, on, or under a sensing region any suitable type of sensors for determining pressure exerted upon the sensing region. In one example, the pressure sensors 224 and 226 include capacitive sensors that can measure a capacitance, or a change in capacitance, caused by a user's touch or proximity of touch, as is common in touchscreen technologies. The pressure sensors 224 and 226 can generate sensor data indicative of a touch or pressure. The sensor data can include a binary indicator that indicates the presence and/or absence of a touch on the sensor. For instance, a “1” for sensor data can indicate that a pressure is sensed at the pressure sensor, and a “0” for the sensor data can indicate that a pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate an amount of pressure above a maximum threshold is detected at the sensor.

The pressure sensors 224 and 226 are illustrated in FIG. 2 as ellipses as an example only. The pressure sensors 224 and 226 generally can be of any suitable shape and size and generate sensor data indicating pressure at any suitable number of points. For instance, in one example, the pressure sensors 224 cover an exterior surface of the lens 216 of the scanner 104 and can be used to determine when the scanner 104 is placed against or in contact with a patient. Additionally or alternatively, the pressure sensors 226 can substantially cover the enclosure 202 of the scanner 104 and can be used to determine when a clinician grabs the scanner 104 for use in an ultrasound examination (e.g., the clinician has a suitable grip on the scanner 104 to perform the ultrasound examination). In some implementations, the pressure sensors 226 cover a portion of the enclosure, such as a handle or a portion of the handle, where the clinician grips or holds the scanner 104 with their hand. The ultrasound system can use the sensor data from one or both of the pressure sensors 224 and 226 to generate a trigger signal that can be used for managing ultrasound devices. For instance, when the sensor data from one or both of the pressure sensors 224 and 226 is above a threshold level, and/or the sensor data from the pressure sensors 226 indicates a grip pattern indicative of a human operating the scanner, the system can generate a trigger signal. The trigger signal can be used to cause the ultrasound system to enable one or more machine-learned models. In some implementations, the trigger signal can be used to cause the scanner 104 to automatically initiate scanning to generate ultrasound data.

In embodiments, the scanner 104 includes an inertial measurement unit (IMU) 228 for generating positional data that determines a position and orientation of the scanner 104 in a coordinate system (e.g., a coordinate system 230 in FIG. 2). The IMU 228 can include a combination of accelerometers, gyroscopes, and magnetometers and can generate positional data including data representing six degrees of freedom (6DOF), such as yaw, pitch, and roll angles in the coordinate system. Typically, 6DOF refers to the freedom of movement of a body in three-dimensional space. For example, the body is free to change position as forward/backward (surge), up/down (heave), and left/right (sway) translation in three perpendicular axes, combined with changes in orientation through rotation about the three perpendicular axes, often termed yaw (normal axis), pitch (transverse axis), and roll (longitudinal axis). Additionally or alternatively, the ultrasound system can include a camera and fiducial markers on the scanner 104 (not shown in FIG. 2) to determine the positional data for the scanner 104. In one example, the system generates, based on the positional data, a trigger signal as described above. For instance, the positional data can indicate that the scanner 104 is within a threshold distance of the patient and the trigger signal can be used by the ultrasound system to enable one or more machine-learned models, such as a machine-learned model to identify and/or segment an anatomy in an ultrasound image.

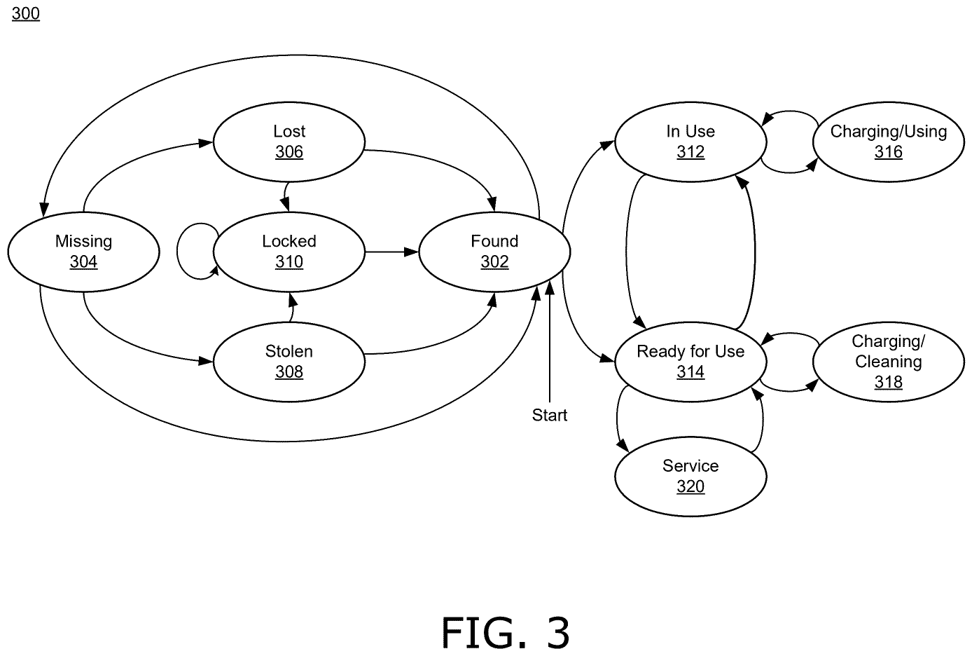

A trigger signal generated by the system (e.g., due to pressure data and/or positional data as described above) can be used to determine a state of the scanner 104 (discussed in more detail with respect to FIG. 3). For example, a trigger signal caused by a human grip on the scanner 104 can be used by the system as a condition (e.g., in combination with other conditions) to enter a state indicative of the scanner 104 being “in-use.” In an example, a trigger signal caused by a human grip on the scanner 104 from the sensors 226 and by pressure against a patient sensed by the sensor 224 can be used by the system as a condition to enter a state indicative of the scanner 104 being “in-use”. In embodiments, a lack of a trigger signal due to pressure data in a time window (e.g., in the last 36 hours) can be used as a condition (e.g., in combination with other conditions) to enter a state indicative of the scanner 104 being “missing.”

In embodiments, the system uses IMU data from the scanner 104 to pair the scanner 104 with the ultrasound machine 102. For instance, a user can move (shake, sweep, arc, etc.) the scanner 104 in the air to perform a gesture, such as by making an “X” pattern, an “O” pattern, and “S” pattern, etc., in the air with the scanner 104. The IMU 228 generates IMU data based on the movement of the scanner 104 relative to the coordinate system. The IMU data from the scanner 104 can be used by the system to read the gesture, and, in response to the gesture, the system can pair the scanner 104 with the ultrasound machine 102, such as via a wireless communication link implemented via the coupling 210. Pairing the scanner 104 and the ultrasound machine 102 can include communicatively coupling the scanner 104 and the ultrasound machine 102 to enable them to be used together to perform an ultrasound examination. For example, the ultrasound machine 102 can configure the scanner 104 to generate ultrasound according to an imaging mode (e.g., B-mode imaging) and the scanner 104 can provide captured ultrasound data to the ultrasound machine 102 to generate an ultrasound image from the ultrasound data.

Inset 232 illustrates a common problem with ultrasound devices, which is exacerbated for ultraportable devices that are small, handheld, and portable. Ultrasound devices, particularly ultraportable devices such as wireless scanners (e.g., wireless probes), have monetary value and can be easy to conceal due to their small form factor. Hence, and unfortunately, ultrasound devices are frequently stolen from care facilities. In other cases, the ultrasound devices are misplaced and ultimately lost. Equipment administrators are typically unsure if a missing ultrasound device is lost or stolen and, thus, are unsure if or when to replace the device. Therefore, the ultrasound devices may not be available when needed for patient care, resulting in the patient not receiving the best care possible. The techniques disclosed herein for managing ultrasound devices provide a way to monitor and manage ultrasound devices such that equipment administrators can be certain if a device is lost, stolen, found, ready for use, in use, being serviced, being charged, being cleaned, etc.

FIG. 3 illustrates an example state machine 300 for managing ultrasound devices in accordance with one or more of the disclosed implementations. The state machine 300 can be used for managing any suitable ultrasound device, including an ultrasound scanner, an ultrasound machine, an ultrasound cart, an ultrasound charging station, etc., such as the ultrasound devices disclosed herein.

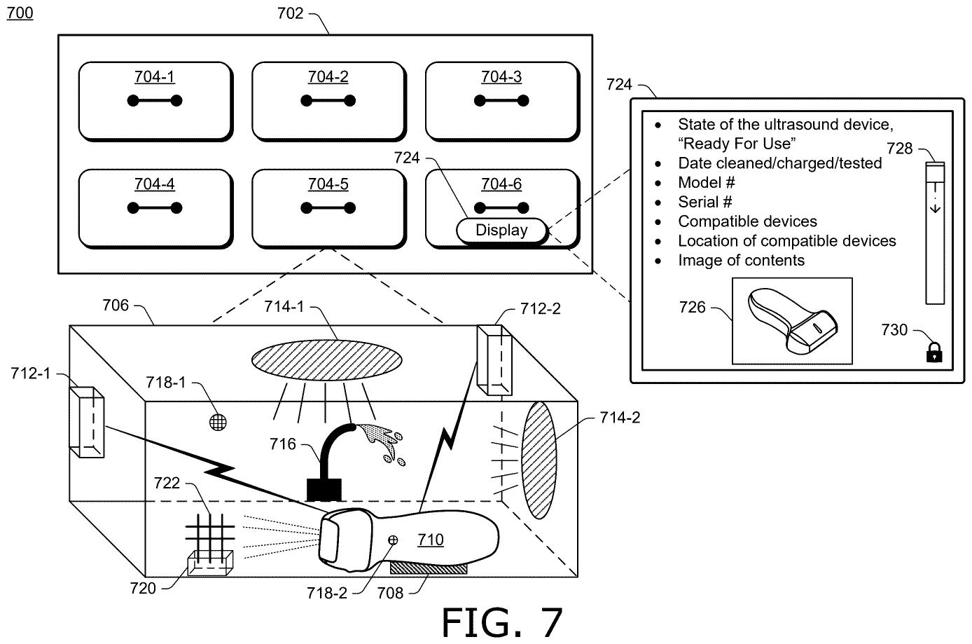

The state machine 300 includes a found state 302, a missing state 304, a lost state 306, a stolen state 308, a locked state 310, an in-use state 312, a ready-for-use state 314, a charging/using state 316, a charging/cleaning state 318, and a service state 320. Example characteristics of these states include the following:
Found state 302. In the found state 302, a location of the ultrasound device is known and the ultrasound device is not missing. For example, a location of the ultrasound device within the care facility may be known, including a room number, a global navigation satellite systems (GNSS) location such as a global positioning signal (GPS) location (e.g., geotag data), another device connected to the ultrasound device and a location of the other device, etc. A current user of the ultrasound device may also be known. The in-use state 312, the ready-for-use state 314, and the service state 320 are sub-states of the found state 302.
Missing state 304. In the missing state 304, the location of the ultrasound device is unknown. It may not yet be known if the ultrasound device is misplaced (e.g., lost) or stolen. The lost state 306, stolen state 308, and locked state 310 are sub-states of the missing state 304.
Lost state 306. In the lost state 306, the ultrasound device is declared missing, and a determination has been made that the ultrasound device has not been stolen. For instance, the ultrasound device has been misplaced, is not where it is expected to be, or is malfunctioning so that its location cannot be determined. The determination that the ultrasound device has not been stolen can be made based on various conditions, which are described in detail below.
Stolen state 308. In the stolen state 308, the ultrasound device is missing and a determination has been made that the device has been stolen, rather than merely lost (e.g., misplaced). For instance, GPS data may be available for the ultrasound device and may indicate that the ultrasound device has been removed from the care facility and is not in possession of an expected user. The determination that the ultrasound device has been stolen can be made based on various conditions, which are described in detail below.
Locked state 310. In the locked state 310, the ultrasound device is missing and has been disabled to cause it to be rendered unsuitable for its intended purpose. For instance, an ultrasound scanner can be disabled by rendering its transducer array inoperable. The ultrasound device can be configured remotely to be locked and/or can configure itself to be locked. When locked, the ultrasound device cannot be used for its intended purpose.
In-use state 312. In the in-use state 312, the ultrasound device's location is known and the ultrasound device is currently being used by an operator, such as during an ultrasound examination. The ultrasound device can be in use when, for example, the scanner is transmitting/receiving ultrasound signals, the ultrasound machine is generating and displaying an ultrasound image, etc. It may be known what devices the ultrasound device is connected to. For instance, it may be known what ultrasound scanner is connected to an ultrasound machine, and the location of the ultrasound scanner can be used to determine or verify the location of the ultrasound machine. The in-use state 312 is a sub-state of the found state 302. The scanner 104 in FIG. 1 used during an ultrasound procedure is an example of an ultrasound device that is in the in-use state 312.
Ready-for-use state 314. In this state, the ultrasound device's location is known and the ultrasound device is in a configuration such that it can be used to treat a patient. For instance, the ultrasound device is cleaned and disinfected and its batteries have sufficient charge to perform an ultrasound examination. In another example, the ultrasound scanner can be powered on but not transmitting/receiving ultrasound signals (e.g., the scanner is being held by the operator but not performing a scan, the scanner is powered on and connected to the ultrasound machine but not performing a scan). The ultrasound device can be maintained in a storage facility, room, or container until it is selected for an examination and thus be in the ready-for-use state 314 while at the storage facility, room, or container. One example of an ultrasound device that has been cleaned, charged, and tested, and is therefore in the ready-for-use state 314, is described with respect to FIG. 7.
Charging/using state 316. In the charging/using state 316, the ultrasound device is currently in use and is also being charged. The location of the ultrasound device is

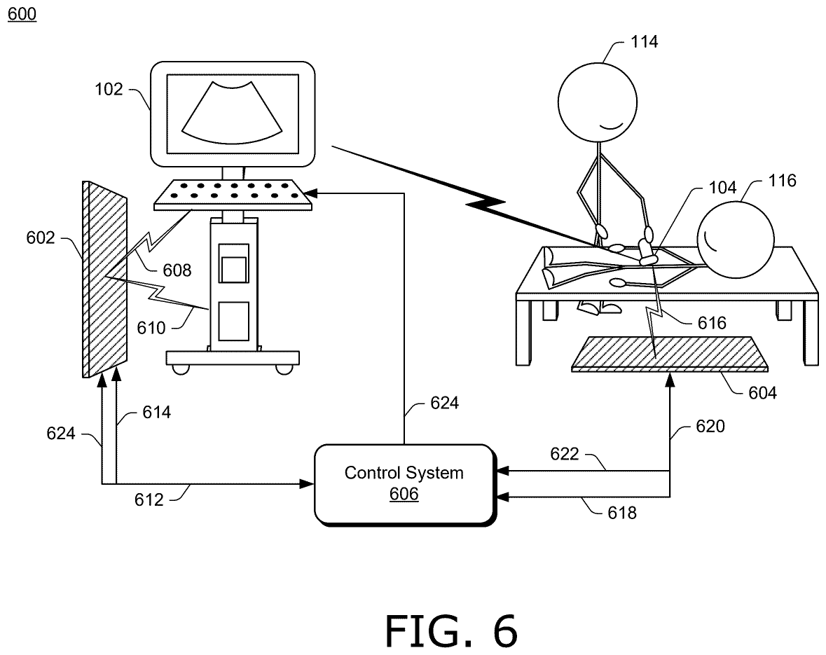

known, such as via a charger that is providing a charging signal to the ultrasound device and/or another ultrasound device that is coupled to the ultrasound device to configure the device for use. An example of a scanner that is currently in use during an ultrasound examination and is also being charged, and is therefore in the charging/using state 316, is described with respect to FIG. 6. The charging/using state 316 is a sub-state of the in-use state 312.
Charging/cleaning state 318. In the charging/cleaning state 318, the location of the ultrasound device is known and the ultrasound device is currently being at least one of cleaned, charged, or tested. One example of an ultrasound device that is being cleaned, charged, and/or tested, and is therefore in the charging/cleaning state 318, is described with respect to FIG. 7. Once the cleaning/charging/testing steps are completed, the ultrasound device can be stored and the state of the ultrasound device can be changed to the ready-for-use state 314.
Service state 320. In the service state 320, the ultrasound device has been removed from availability for use so that it can be serviced. For example, the ultrasound device can be returned to its manufacturer for warranty service work. Once the repair work is completed, the ultrasound device can be returned to the owner and the state of the ultrasound device can be changed from the service state 320 to the ready-for-use state 314.

The state machine 300 can implement a protocol to determine a state of the ultrasound device based on a combination of conditions and initiate appropriate actions for the states, once entered. For instance, if the device is lost, the state machine 300 can initiate one or more actions to recover it; if the device is stolen, the state machine 300 can initiate one or more additional actions to disable the device and remove at least some of the device's economic value. The state transitions of the state machine 300 can be determined by combinations of conditions associated with each of the states. In aspects, the combinations can include conditions across the states.

Example conditions of the states including the missing state 304, the lost state 306, and the stolen state 308 that can be used in combination to enter one of these states are described as follows. Some example conditions for the missing state 304 include loss of a communication link, the ultrasound device being no longer connected to a local network of the care facility, the ultrasound device being no longer connected to the ultrasound machine, a user reporting that the ultrasound device is missing, or the ultrasound device not being located via a geotag (GPS location). Some example conditions for the lost state 306 include an associated geotag indicating an unexpected location in the care facility, the ultrasound device not being “seen” or detected by any other device in the care facility, the ultrasound device having been signed out by a user but not being located with that user, or a user reporting that the ultrasound device is not where the user left it. Some example conditions for the stolen state 308 include an associated geotag indicating that the ultrasound device is outside of the care facility (or use area, e.g., outside a geofence area) or the ultrasound device moving in an unfamiliar way—a rate of movement, an orientation of movement (put into a person's pocket, based on a grip map (e.g., fingerprint detection, fingerprint not identified to the scanner), based on device IMU data, etc.). Additional example conditions for the stolen state 308 include the device IMU data being mapped to one or more gestures indicative of stolen devices (used in a non-standard manner, taken apart, etc.), an unexpected fingerprint read by a grip map, detection of the battery being removed from the ultrasound device, the ultrasound device (e.g., scanner) attempting to pair with a new display device, or the ultrasound device being determined to be in a Faraday bag (e.g., device does not recognize any other devices in the care facility but its location data suggests that the ultrasound device is located in the care facility).

The state transitions of the state machine 300 into the missing state 304, the lost state 306, and/or the stolen state 308 can be determined by combinations of the conditions above for each of these states, across the states, in various ways including logic operations and/or weighted conditions. In an example of logic operations on the conditions, to transition to the missing state 304, the logic operations can require the occurrence of at least two “Missing” conditions. In another example, to transition from “Missing” to “Lost,” the logic operations can require the occurrence of two or more “Lost” conditions and not a “Stolen” condition. In an example of weighted conditions, weights can be assigned to each condition. For instance, if the sum of weights for conditions that occur is greater than a threshold, the state can transition to another state. In some implementations, a “false positive” condition can be given a sufficiently high weight to automatically transition.

When a state of the state machine 300 has been entered for an ultrasound device, the system can initiate an appropriate action or function associated with that state. Some example actions for the states are described below. For instance, some example actions for the missing state 304 include pinging the ultrasound device with the network of the care facility, causing the missing device to send a ping (e.g., the ultrasound device can include a coin-cell battery to power an embedded geotag), retrieving a last-known location of the ultrasound device via an associated geotag or geofence data (e.g., a last known location of the ultrasound device based on a network node that communicated with the ultrasound device), and/or contacting a last-known user of the ultrasound device and determining if the ultrasound device was loaned to another person. For the lost state 306, some example actions for the state machine 300 include causing the ultrasound device to emit an alarm (haptic, visual (e.g., light, display), audible, etc.), sending an alert to staff, notifying an equipment administrator, securing a replacement device, posting an incentive for the return of the lost device, sending a request via the ultrasound device for a user to enter an ID (username, password, passcode, etc.), and/or starting a timer to enter the locked state 310 if the ultrasound device is lost for more than a threshold amount of time. In implementations, different threshold amounts of time can be used for different devices. For example, the state machine 300 can use a first threshold amount of time for a device having a higher priority level, such as a multi-array scanner, and a second threshold amount of time for another device having a lower priority level, where the first threshold amount of time is less than the second threshold amount of time. Accordingly, using different priority levels and different corresponding time thresholds enables the state machine 300 to enter particular states (the missing state 304, the lost state 306, the stolen state 308, etc.) and initiate corresponding actions sooner for devices having higher priority levels than other devices with lower priority levels.

For the stolen state 308, one example action includes sending a notification (e.g., warning message) indicating that the ultrasound device can function for only “X” more scans (e.g., 10 more scans), “X” more days (e.g., three days), etc. before being locked, unless an ID is entered. This can enable the ultrasound device to continue to be used during that time for potentially life-saving examinations or procedures. Additional example actions for the stolen state 308 include obtaining a location of the ultrasound device (e.g., via an embedded geotag or application on a smartphone if the ultrasound device is paired to the smartphone), notifying a manufacturer of the ultrasound device, notifying authorities of the stolen device, removing some of the economic value of the ultrasound device (e.g., by removing firmware, formatting memory), sending instructions via the ultrasound device for its return, and/or monitoring online auctions (e.g., eBay™) for a sale of the lost device. For the found state 302, example actions include sending a pre-paid postage box for return if the ultrasound device is found outside of the care facility, notifying the equipment administrator, unlocking the device if it was in the locked state 310, and/or canceling a replacement order. For the service state 320, example actions include preparing the device for repair such as by disabling tethering (e.g., geotag tracking) for the device, notifying a manufacturer of the device of the service, and/or placing a shipping order to send the device to a service center or the manufacturer for repair.

One advantage of a system that implements the state machine 300 is that the system can determine when a missing ultrasound device is lost versus stolen. In addition, state transitions of the state machine 300 are defined based on combinations of conditions, which reduces the likelihood of a false positive. Further, the use of the state machine 300 enables fast recovery of lost devices and reduces incentives for theft of devices. The state machine 300 can also be easily implemented as part of asset tracking and can help address the common question of “Where's my ultrasound device?” Moreover, use of the state machine 300 increases the likelihood that an ultrasound device is “ready for use,” so patients receive better care compared to conventional asset tracking systems.

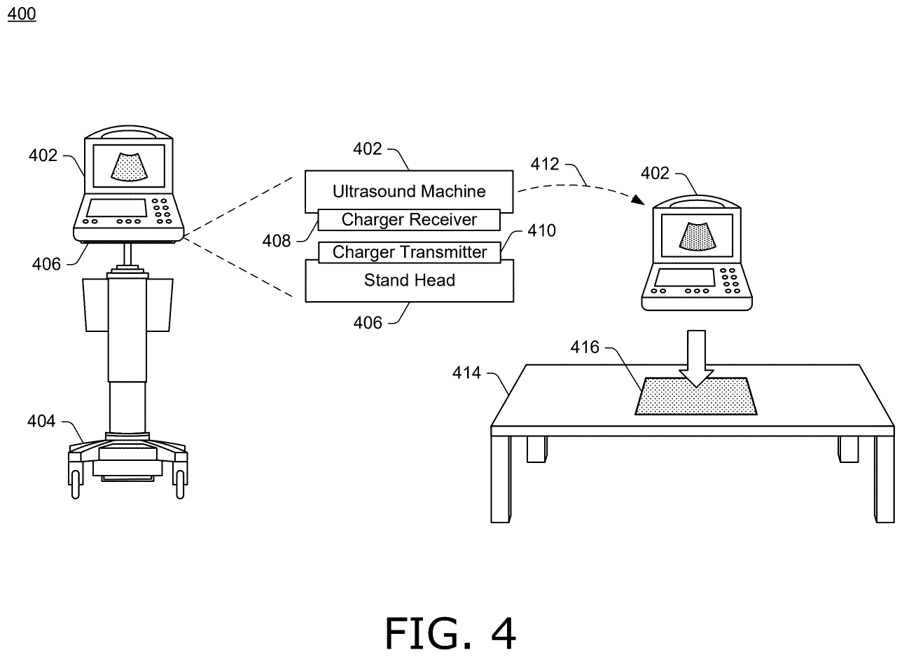

FIG. 4 illustrates an example system 400 for managing ultrasound devices, including charging of ultrasound devices. The system 400 includes an ultrasound machine 402 (e.g., the ultrasound machine 102) and an ultrasound cart 404 (e.g., the ultrasound cart 120) having a stand head 406 on which the ultrasound machine 402 is mountable in a first configuration. The ultrasound machine 402 is coupled to a charger receiver 408. For instance, the charger receiver 408 can be attached to a bottom side of the ultrasound machine 402 that faces the stand head 406. The stand head 406 is coupled to a charger transmitter 410. For instance, the charger transmitter 410 can be attached to a top side of the stand head 406 that faces the ultrasound machine 402. In this first configuration where the ultrasound machine 402 is mounted on the stand head 406, the charger receiver 408 and the charger transmitter 410 can implement a non-contact charging system (e.g., wireless charging system) that charges a battery of the ultrasound machine 402. For example, the non-contact charging system can implement inductive charging, radio frequency (RF) or resonance charging, optical charging, etc. The ultrasound cart 404 can include a battery and/or be connected to a power source, such as a wall outlet, to provide power to the charger transmitter 410 that wirelessly couples charge to the charger receiver 408.

In a second configuration, the ultrasound machine 402 is removed from the stand head 406 of the ultrasound cart 404, as indicated by an arrow 412, and placed on a mounting surface 414. The mounting surface 414 can include a table, desk, bench, countertop, wall, etc., and includes an additional charger transmitter 416. For instance, the additional charger transmitter 416 can be mounted on or in the mounting surface 414. Additionally or alternatively, the charger transmitter 410 can be removed from the stand head 406 and placed on or in the mounting surface 414 to form the additional charger transmitter 416. In this second configuration where the ultrasound machine 402 is placed on the mounting surface 414, the charger receiver 408 and the additional charger transmitter 416 can implement a non-contact charging system (e.g., wireless charging system) that charges a battery of the ultrasound machine 402. For example, the non-contact charging system can implement inductive charging, RF or resonance charging, optical charging, etc. The mounting surface 414 can include a battery and/or be connected to a power source, such as a wall outlet, to provide power to the additional charger transmitter 416 that wirelessly couples charge to the charger receiver 408.

Hence, the system 400 can implement non-contact charging mechanisms (resonance, induction, optical, etc.) that support charging of an ultrasound device in different configurations (e.g., when an ultrasound machine is on a stand head and when the ultrasound machine is placed on a table). When charging in one of these configurations, the location of the ultrasound device can be determined by the system (e.g., via the location of a charger transmitter that is providing a charging signal to the ultrasound device). The location (or lack of a location) can be used by the state machine 300 to determine a state of the ultrasound device. In implementations, the system 400 determines the location of the ultrasound device based on a device identifier of the ultrasound device and a geotag location of the charger transmitter providing charge to the ultrasound device.

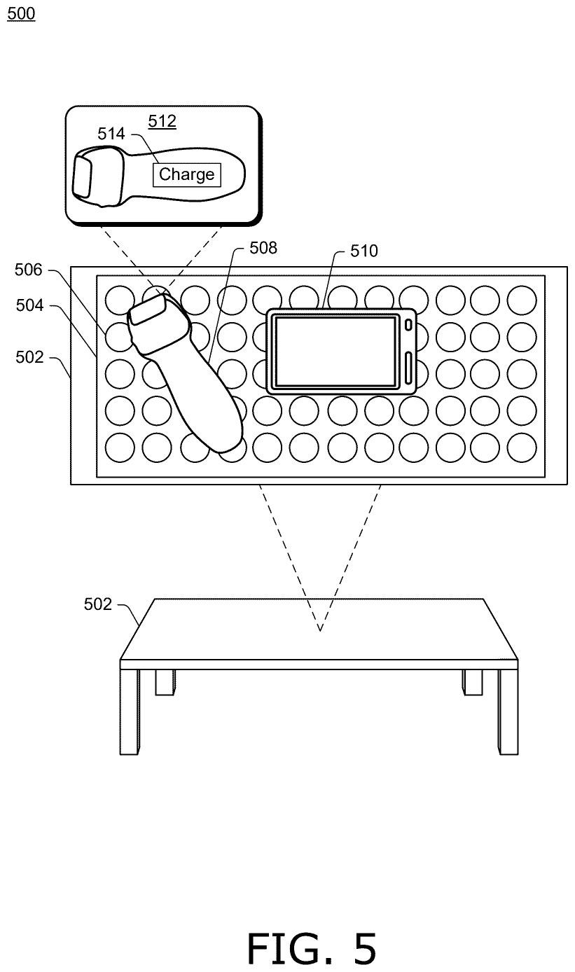

FIG. 5 illustrates an example system 500 for managing ultrasound devices, including charging of ultrasound devices. The system 500 can be configured to control simultaneous charging of multiple ultrasound devices. The system 500 includes a surface 502 that can include any suitable surface, including a surface of a table, desk, shelf, countertop, ultrasound cart, etc. Embedded in or on the surface 502 is a charger array 504 that includes an array of charger transmitters 506. Each of the charger transmitters 506 can be configured for non-contact charging (e.g., inductive charging, RF or resonance charging, optical charging, and the like). The charger transmitter 410 is an example of the charger array 504 that includes the charger transmitters 506.

In FIG. 5, ultrasound devices placed on the charger array 504 include an ultrasound scanner 508 and a computing device 510. The computing device 510 can include a smartphone or tablet having an ultrasound application installed that configures the computing device 510 as an ultrasound machine that can be coupled to the ultrasound scanner 508. The ultrasound scanner 508 is an example implementation of the scanner 104. The charger array 504 can configure the charger transmitters 506 to charge the ultrasound scanner 508 and the computing device 510, which can include charger receivers (e.g., the charger receiver 408) to receive the charge from the charger transmitters 506. While being charged, the ultrasound scanner 508 and the computing device 510 are examples of ultrasound devices in the charging/cleaning state 318 of the state machine 300 in FIG. 3.

The charger array 504 can implement a charging control system to configure the charger transmitters 506 to charge the ultrasound scanner 508 and the computing device 510. In embodiments, the charging control system can efficiently charge the ultrasound devices (e.g., the ultrasound scanner 508 and the computing device 510) in a smart manner. For instance, the charger array 504 can detect which of the charger transmitters 506 are proximate to an ultrasound device and activate those transmitters to provide charge to the device. In an embodiment, the charger array 504 determines a plurality of transmitters that are proximate to an ultrasound device (e.g., underneath it) and activates each of the transmitters of the plurality for a duration in a round-robin fashion. The duration can be a prescribed length of time (e.g., two minutes, five minutes, ten minutes).

Alternatively, the duration can be based on a parameter or component of the system 500 and adjusted dynamically to efficiently and safely charge the device. For instance, the duration can be based on heat sensed at the ultrasound device and/or the charger transmitters 506, an amount of power transferred, an amount of charge on the ultrasound device to enable an amount of scan time, etc. The order of transmitters selected to charge the device can be selected to reduce heat. Hence, the system 500 can efficiently charge multiple ultrasound devices simultaneously so that the devices are ready for use. Once the device is charged by the system 500, the device can be assigned to the ready-for-use state 314 of the state machine 300 in FIG. 3.

At inset 512 in FIG. 5, the ultrasound scanner 508 is illustrated in an orientation that exposes an underside of the ultrasound scanner 508 (e.g., relative to the orientation of the ultrasound scanner 508 when it is placed on the charger array 504). In the orientation illustrated at inset 512, the underside of the ultrasound scanner 508 includes a region 514 that includes a visual representation indicating where to charge the ultrasound scanner 508. For instance, the region 514 can include text that says “charge here,” an icon indicating that the region 514 is a target location on the ultrasound scanner 508 for where to apply charge (e.g., a bullseye with a lightning bolt), etc. The region 514 indicates to a user how to place the ultrasound scanner 508 on the charger array 504 for non-contact charging.

FIG. 6 illustrates an example system 600 for managing ultrasound devices, including charging of ultrasound devices. The system 600 includes a first charger array 602 and a second charger array 604. The charger arrays 602 and 604 are examples of the charger array 504 and the charger transmitter 410 and can include any suitable number of charging elements in any suitable configuration. In the illustrated example, the first charger array 602 is mounted vertically (e.g., on or in a wall, on an ultrasound cart, on a utility cart) Further, the second charger array 604 is mounted horizontally (e.g., on or in a floor, on or in a table (e.g., an examination table), on or in a patient's bed, etc.). The charger arrays 602 and 604 are coupled to a control system 606 implemented to control the charger arrays 602 and 604 and determine a position (e.g., location) of an ultrasound device based on charger transmitters in the arrays that provide charge to the ultrasound device.

In the example in FIG. 6, the first charger array 602 provides charge 608 to the ultrasound machine 102 and charge 610 to an ultrasound cart on which the ultrasound machine 102 is mounted. For instance, the ultrasound cart can house a spare battery for the ultrasound machine 102, and the charge 610 from the first charger array 602 can charge the spare battery. Hence, the control system 606 receives charging status data 612 from the first charger array 602 and provides charging control data 614 to the first charger array 602. The charging status data 612 can include any suitable data indicative of a charging status of a device being charged by the first charger array 602, data indicative of a charger transmitter of the first charger array 602, and the like. The charging control data 614 can include any suitable data to instruct the first charger array 602 to charge an ultrasound device, including a charging waveform, charging profile, selection of one or more charger transmitters in the first charger array 602, etc.

Further illustrated in FIG. 6, the second charger array 604 is providing charge 616 to the scanner 104, which can be wirelessly coupled to the ultrasound machine 102. The scanner 104 can receive the charge 616 as long as the scanner 104 is within a threshold distance from the mounting surface or from the second charger array 604, where the distance is measured perpendicular to the mounting surface or the second charger array 604. Within the threshold distance, the scanner 104 can be coupled to the second charger array 604. Based upon the charge 616 coupled to the scanner 104 via the charger transmitters of the second charger array 604, the system 600 can determine a position (relative to the second charger array 604) of the scanner 104. Hence, the first charger array 602 provides scanner position data 618 (more generally, ultrasound device position data) to the control system 606. Similar to the control of the first charger array 602, the control system 606 also provides charging control data 620 to the second charger array 604 and receives charging status data 622 from the second charger array 604. The charging status data 622 can include any suitable data indicative of a charging status of a device being charged by the second charger array 604, data indicative of a charger transmitter of the second charger array 604, and the like. The charging control data 620 can include any suitable data to instruct the second charger array 604 to charge an ultrasound device, including a charging waveform, charging profile, selection of one or more charger transmitters in the second charger array 604, etc.

The control system 606 can process the scanner position data 618 from the second charger array 604 and generate guidance data 624. The control system 606 can provide the guidance data 624 to the ultrasound machine 102 in any suitable way, such as via a connection to the ultrasound machine 102. In an embodiment, the ultrasound machine 102 includes the control system 606 (e.g., the ultrasound machine 102 implements the control system 606 on the processors 106). Additionally or alternatively, the control system 606 can provide the guidance data 624 to the ultrasound machine 102 via the first charger array 602. For example, the control system 606 can provide the guidance data 624 to the first charger array 602, which can modulate the guidance data 624 onto the charge 608 to communicate the guidance data 624 to the ultrasound machine 102. The guidance data 624 can include any suitable data regarding the location of the scanner 104, including a visual representation of the scanner 104 relative to a patient anatomy, an orientation of the scanner 104 in a coordinate system, an indication showing how to move the scanner 104 to better image a patient anatomy, etc., and the ultrasound machine 102 can display the visual representation.

While being charged and used to examine the patient 116 in FIG. 6, the ultrasound machine 102 and the scanner 104 are in the charging/using state 316 of the state machine 300 in FIG. 3. The control system 606 can instruct a charger (e.g., the second charger array 604) to charge an ultrasound device (e.g., the scanner 104) based on any suitable data, including the patient's history, the patient's current condition and/or diagnosis, a battery life/scan time remaining of the scanner 104, an examination type, a schedule or calendar for the ultrasound device (e.g., more charge may be applied to the ultrasound machine 102 if it is scheduled to be used in another ultrasound examination for another patient following this current examination for the patient 116).

In embodiments, the scanner 104 includes an IMU (e.g., the IMU 228) that generates IMU data (positional data the determines a position and orientation of the scanner 104 in a coordinate system), and the user 114 can perform a gesture with the scanner 104 to enable or disable charging of the scanner 104 via the second charger array 604. For instance, the system 600 can use IMU data for a first gesture to enable charging of the scanner 104 and use IMU data from a second gesture to disable the charging of the scanner 104. Other examples are described above with respect to FIG. 2.

In embodiments, one or more of the charger arrays 602 and 604 and the scanner 104 implement circuitry for electromagnetic leakage reduction, including combined shielding and heat management, as described in U.S. application Ser. No. 18/125,575 filed on Mar. 23, 2023, and entitled Ultrasound Probe with Thermal Management to Aliakbari, the disclosure of which is incorporated herein by reference in its entirety.

FIG. 7 illustrates an example system 700 for managing ultrasound devices, including charging, cleaning, and testing of ultrasound devices. The system 700 includes a container 702 that includes drawers 704-1 through 704-6. The container 702 can be located in any suitable location in a care facility where ultrasound systems are used, such as a nursing station, ultrasound storage room, etc. In implementations, the drawers 704 are containers that can be sealed to prevent their contents from being exposed to an environment outside of the drawers 704. The drawers 704 can be used to simultaneously charge, clean, and test an ultrasound device, such as an ultrasound scanner, and while doing so, the device is in the charging/cleaning state 318 of the state machine 300. When the device is charged, cleaned, and tested, the drawers 704 can store the device so that it is ready for use (e.g., the device is in the ready-for-use state 314 of the state machine 300). The charging, cleaning, and/or testing of the ultrasound device can be referred to as a maintenance cycle. The maintenance cycle can include charging, cleaning, and testing cycles collectively or individually.

The contents of one of the drawers 704 configured to simultaneously charge, clean, and test an ultrasound device are illustrated in FIG. 7 as example contents 706. The contents 706 include a mount 708 configured to securely hold an ultrasound device (e.g., an ultrasound scanner 710) within the drawer 704. The mount 708 can be a multi-purpose mount, which can securely hold the ultrasound device while at the same time performing at least one additional function. For instance, the mount 708 can include a charger array or charger transmitter that transfers charge to a battery of the ultrasound scanner 710 (e.g., the scanner 104, the ultrasound scanner 508). Additionally or alternatively, the mount 708 can provide instructions to the ultrasound scanner 710 to operate the ultrasound scanner 710 according to a calibration routine and/or a fault check (described below in more detail). Hence, the mount 708 can be coupled to a user-operated computing device (not shown in FIG. 7 for clarity) to control the mount 708, or the mount 708 itself can include a computing device.

The contents 706 also include charger transmitters 712-1 and 712-2 implemented to provide wireless charging signals to the ultrasound device (e.g., the ultrasound scanner 710). The charger transmitters 712-1 and 712-2 are examples of the charger arrays 602 and 604, the charger array 504, and the charger transmitter 410. The contents 706 also include cleaning sources 714-1 and 714-2 that are implemented to emit a light (ultraviolet radiation, gamma rays, etc.) to clean and disinfect the ultrasound scanner 710. The contents 706 also include a fluid source 716 implemented to emit a fluid into the drawer 704. The fluid can include a disinfectant fluid (gel, foam, liquid, gas, etc.) to clean and disinfect the ultrasound scanner 710. The fluid can also include a rinsing fluid (e.g., water) for a pre-cleaning cycle to rinse off dirt and debris prior to cleaning and disinfecting and/or for a post-cleaning cycle to rinse off aggressive disinfectants. The system 700 can apply the fluid from the fluid source 716, the light from the cleaning sources 714-1 and 714-2, and the charge from the charger transmitters 712-1 and 712-2 in any suitable order or timing, including simultaneously or at non-overlapping times.

In an example, when simultaneously charging the ultrasound scanner 710, via the mount 708 and/or the charger transmitters 712-1 and 712-2, and cleaning the ultrasound scanner 710, with fluid from the fluid source 716, the system 700 has the added benefit that the fluid can remove heat from the ultrasound scanner 710 that is caused by the charging. Hence, the ultrasound scanner 710 can be charged more quickly than conventional charging systems and thus be ready for use before an ultrasound scanner charged by the conventional charging system. In an example, the cleaning fluid cools the ultrasound scanner 710 to a temperature that is at or below a threshold temperature, such as an ambient temperature outside of the drawer 704. Such temperature becomes an initial temperature of the ultrasound scanner 710 when a clinician removes the ultrasound scanner 710 from the drawer 704 to use it to perform an ultrasound scan of a patient. During use, the temperature of the ultrasound scanner 710 increases due to heat from the patient when touching the patient's body and heat generated by the ultrasound scanner 710 itself. The temperature of the ultrasound scanner 710 can continue to increase until reaching a critical temperature threshold (e.g., 48° C.) that triggers thermal shutdown, which is implemented to prevent harming the patient. Accordingly, starting the scan with the scanner temperature at or below the ambient temperature, compared with conventional systems that start at a scanner temperature greater than the ambient temperature (e.g., due to charging), increases the scan time available for the ultrasound scanner 710 before the ultrasound scanner 710 reaches thermal shutdown because the temperature range in which the ultrasound scanner 710 is usable (e.g., from the initial temperature to the critical temperature threshold) is greater than that of the conventional systems. Accordingly, when using the system 700, patients can receive better care compared to the use of conventional charging systems at least.

In implementations, the scanner temperature refers to the temperature of the exterior surface of the ultrasound scanner 710. One or more sensors 718 can be used to detect and/or measure the scanner temperature. For example, an infrared (IR) camera 718-1 can be implemented in the drawer 704 to detect and monitor the scanner temperature during the charging, cleaning, testing, and storing of the ultrasound scanner 710. The one or more sensors 718 can include thermal sensors 718-2 (e.g., thermistors) implemented on the ultrasound scanner 710, which enable the ultrasound scanner 710 to detect its own temperature and communicate (e.g., transmit) its temperature to the system 700. In one example, the IR camera 718-1 can be used to verify the temperature detected by the thermal sensors 718-2, which can provide redundancy to catch potential errors (calibration, bias, drift, etc.) by the thermal sensors 718-2.

The contents 706 can also include a calibration tool holder 720 and a calibration tool 722. The calibration tool 722 can include one or more wires (e.g., arranged in a grid) that are held in a position by the calibration tool holder 720. The system 700 can enable the fluid source 716 to dispense fluid to submerge the calibration tool 722 and the ultrasound scanner 710. The system 700 can then run a calibration routine on the ultrasound scanner 710 to check for faults and adjust parameters according to data acquired during the calibration routine. For instance, the system 700 can instruct, via the mount 708, the ultrasound scanner 710 to transmit ultrasound at the calibration tool 722 and generate image data based on reflections of the ultrasound from the calibration tool 722. The mount 708 can include a processor system, or access a processor system of another computing device, to process the image data to determine if the ultrasound scanner 710 is in calibration or if it needs adjustment. To do so, the processor system can compare the resolution in the image data of the calibration tool 722 to a threshold resolution. In an example, the processor system implements a machine-learned model to process the image data and determine a calibration result that indicates an amount of adjustment needed to bring the ultrasound scanner 710 into calibration.

In embodiments, the system 700 implements a fault check on the array elements of the ultrasound scanner 710. For instance, the system 700 can remove fluid from the drawer 704 that was introduced via the fluid source 716 (e.g., via a drain and/or pump, not shown for clarity) and apply test signals to the ultrasound scanner 710 via the mount 708. The test signals can be used to determine an amount of capacitance across array elements of the transducer in the ultrasound scanner 710, which can determine if the array elements are faulty. If faulty elements are found, they can be disabled. Alternatively, the system 700 can indicate that the ultrasound scanner 710 is in need of service and change the state in the state machine 300 to the service state 320 for the ultrasound scanner 710. Accordingly, the fault check provides feedback for a service strategy because the fault check indicates that the ultrasound scanner 710 is functioning properly or is in need of service. If the ultrasound scanner 710 is in need of service, the system 700 can initiate a service order for the ultrasound scanner 710 and notify the equipment administrator. In addition, the system 700 can identify another ultrasound scanner, which is in the ready-for-use state 314 of the state machine 300 or will be ready for use soon (e.g., within “X” number of minutes), for the clinician to use.

Hence, the system 700 can simultaneously clean, disinfect, charge, and test an ultrasound device, and place the ultrasound device in the ready-for-use state 314 of the state machine 300. The drawers 704 of the system 700 can include a display 724 to indicate data corresponding to an ultrasound device stored in the drawer, including the state of the ultrasound device in the state machine 300 and a date, timestamp, etc. that the ultrasound device entered the displayed state. The display 724 can also display a model number and serial number of the ultrasound device stored in the drawer 704. The display 724 can also display indications of compatible devices for the ultrasound device stored in the drawer 704, including locations of the compatible devices in the care facility. Examples of compatible devices include an ultrasound machine configured to be paired to a wireless ultrasound scanner stored in a drawer, an ultrasound cable for an ultrasound scanner stored in a drawer, and an ultrasound scanner for a needle guide stored in a drawer. The display 724 can also display an image (a thumbnail image 726, an image captured by a camera in the drawer 704, etc.) of the ultrasound device that is stored in the drawer 704. In embodiments, the display 724 implements a touchscreen, and a user can scroll down (via a drag gesture, via a slider 728, etc.) and/or across the display 724 to access hidden content that is not currently being displayed.

The display 724 can include a security indicator 730 (icon, image, animation, light-emitting diode (LED), text, etc.) that indicates whether the drawer 704 is locked or unlocked. For instance, during the process of charging, cleaning, and testing the ultrasound device located inside the drawer 704, the drawer 704 can be locked to prevent human access and the security indicator 730 can be displayed as a “locked” indication via the display 724 to notify the user that the drawer 704 is currently locked. For example, if the charging, cleaning, and testing process is complete but the temperature of the ultrasound device has not yet cooled to a safe temperature for the clinician to touch, the drawer 704 can remain locked and the security indicator 730 can be displayed to visually notify the clinician that the drawer 704 is locked and unsafe to open.

Once the temperature of the ultrasound device has dropped below a safe temperature, the security indicator 730 can change to an “unlocked” indication to notify the clinician that the drawer 704 is safe to open. In some implementations, the drawer 704 can be automatically unlocked and the security indicator 730 can be changed to an “unlocked” indication once the ultrasound device has cooled to a safe temperature even though the ultrasound device is still cooling to a target temperature (e.g., ambient temperature or below). In this way, the ultrasound device can be ready for use sooner (e.g., before being completely cooled to the target temperature) to be available in an emergency situation, for example.

While in proximity to, or coupled to a charging system as illustrated in FIGS. 4-7, the location of an ultrasound device is known or can be determined. For instance, geolocation (e.g., GPS) and other capabilities that support automated location and spatial positioning of the charger (e.g., the second charger array 604) can be built into the charger and enabled while the ultrasound device is charging or in an “always on” configuration. Device identification, status and health, and communication-data transmission capabilities (e.g., network communications) can be built into chargers and ultrasound devices. These capabilities can be enabled during charging. A device identifier (e.g., device ID) and other information can be transmitted to a database indicating a specific device is being charged by a specific charger. When combined, location and device ID allow a device to be tracked to a charging solution in a known location.

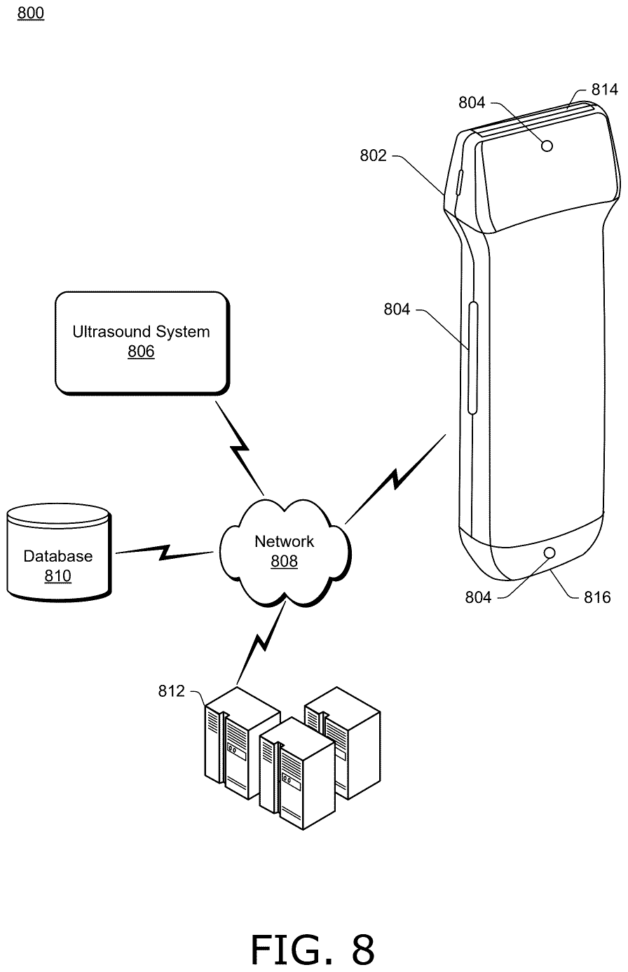

For example, FIG. 8 illustrates an example environment 800 for managing ultrasound devices, including an ultrasound scanner 802 having a lightbar 804, and an ultrasound system 806. The ultrasound scanner 802 (e.g., the scanner 104, the ultrasound scanner 508, the ultrasound scanner 710) and the ultrasound system 806 (e.g., the ultrasound system 100) are meant to be non-limiting examples of ultrasound devices that can be managed according to the disclosed implementations, including being tracked according to the state machine 300 and charged, cleaned, and/or tested with one of the systems 400, 500, 600, and 700. The ultrasound scanner 802 and the ultrasound system 806 are communicatively coupled to a network 808. The network 808 can include any suitable network, such as a local area network, a wide area network, a near field communication (NFC) network, the Internet, an intranet, an extranet, a system bus that couples devices or device components (e.g., in an ASIC, an FPGA, or a system-on-chip (SOC)), and combinations thereof. Accordingly, in embodiments, information can be communicated to the ultrasound scanner 802 and the ultrasound system 806 through the network 808. For instance, a database 810 can store instructions executable by a processor system of the ultrasound scanner 802 and/or the ultrasound system 806 and communicate the instructions via the network 808. Additionally or alternatively, the database 810 can maintain device data, such as device IDs for ultrasound devices that are managed with the state machine 300, and communicate the device data to the ultrasound system 806, a server system 812, or any suitable computing device (e.g., the computing device 510) that can implement an application as part of a system for managing ultrasound devices. For instance, the server system 812 can maintain an asset tracking application that implements the state machine 300 to manage ultrasound devices according to the disclosed implementations.

In embodiments, the database 810 implements a medical archiver (e.g., a VNA) that maintains patient medical records. The medical archiver can store medical data (e.g., ultrasound examination data) generated by the ultrasound system 806 and provide medical data (e.g., data from previous ultrasound examinations) to the ultrasound system 806 for use in a current ultrasound examination.

The server system 812 can be a separate device from the ultrasound system 806. Alternatively, the server system 812 can be included in the ultrasound system 806. In one example, the server system 812 and the database 810 are included in the ultrasound system 806. In an example, the server system 812 is implemented as a remote server system that is remote from (e.g., not collocated with) the ultrasound system 806.

In the illustrated example, the ultrasound scanner 802 includes the lightbar 804. The lightbar 804 can include a light source in the visible spectrum to indicate to a user an orientation to hold the ultrasound scanner 802. For example, the lightbar 804 can serve as a fiducial marker for the ultrasound scanner 802. In embodiments, the lightbar 804 indicates a battery status of the ultrasound scanner 802. For instance, the lightbar 804 can enable one or two light sources to indicate a battery status of less than half capacity and three or four light sources to indicate a battery status of more than half capacity. In an example, the lightbar 804 changes a color of the light emitted to indicate the battery status. The lightbar 804 can be used to illuminate a workspace with visible light, for example, if the ultrasound scanner 802 is being used in a dark or low-light environment (e.g., triage center, battlefield, during a power-outage).

In embodiments, the lightbar 804 can indicate a state of the state machine 300 that the ultrasound scanner 802 is currently in. For example, the lightbar 804 can indicate the ultrasound scanner 802 is in the ready-for-use state 314 based on a number of light sources illuminated, or that the ultrasound scanner 802 is in the missing state 304 based on a different number of light sources illuminated, a color of the light sources, a blinking pattern, and the like.

In embodiments, the lightbar 804 can include one or more light sources outside the visible spectrum that emit non-visible light that can be used to determine a state of the state machine 300 for the ultrasound scanner 802. For instance, a care facility can include sensors that can detect the non-visible light, such as sensors in the walls of the care facility, in light switches, in electric wall outlets, at security gates, included in an ultrasound machine, etc. When a sensor detects light (e.g., non-visible light) emitted by the lightbar 804, the sensor can report the device identifier (which can be embedded into the light that is emitted) and the location of the ultrasound scanner 802 (e.g., the location of the sensor) to a computing device implementing the state machine 300. The state machine 300 can use the location reported by the sensor as one of the conditions to transition to a state of the state machine 300 for the ultrasound device, such as to the found state 302.

Additionally or alternatively, the system can use the location reported by the sensor to take an action for a state of the state machine 300 for the ultrasound scanner 802. For instance, the system can send an alert to security that the ultrasound scanner 802 is not in an expected location, a message to an equipment manager, a message to a doctor who previously reported the ultrasound scanner 802 as lost, and the like. Any ultrasound device (e.g., the ultrasound system 806) that is managed according to the disclosed implementations can include a light source that can emit visible light to report a state of the state machine 300, and/or a light source that can emit non-visible light that can be sensed and used to set a state of the state machine 300. In some aspects, the lightbar 804 can be disposed at any suitable location on a housing of the ultrasound scanner 802, such as on a handle, proximate to a transducer assembly 814 (e.g., transducer assembly 214) of the ultrasound scanner 802, proximate to an end 816 of the handle (e.g., proximal end portion 206) that is opposite the transducer assembly 814, etc.

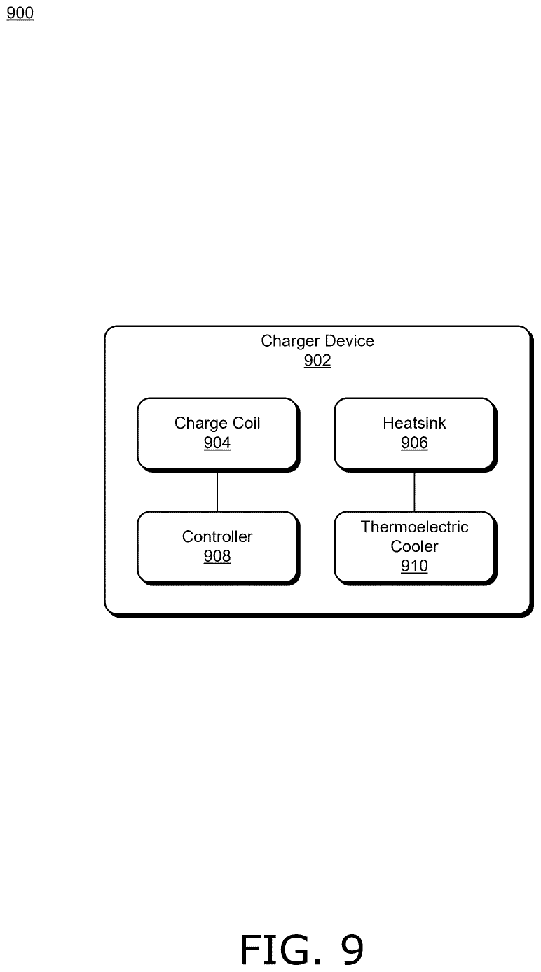

FIG. 9 illustrates a block diagram 900 of an example charger device 902 for simultaneous charging and heat removal. The charger device 902 includes a charge coil 904 and a heatsink 906. In aspects, the charger device 902 also includes a controller 908 electrically connected to the charge coil to control electric current running through the charge coil 904. In some examples, the controller 908 is implemented on a printed circuit board (PCB), such as a main logic board of the charger device 902. When the charger device 902 is connected to a power source, such as line power, the controller 908 can enable electric current (e.g., alternating current (AC)) to pass through the charge coil 904, which creates a magnetic field for magnetically coupling with another device to wirelessly transmit power to the other device. The electric current also causes the charge coil 904 to generate heat.

The charger device 902 can also include a cooling mechanism, such as a thermoelectric cooler 910 to assist the heatsink 906 in drawing heat from the charge coil 904. For example, the thermoelectric cooler 910 can be disposed between the heatsink 906 and the charge coil 904. On one side of the thermoelectric cooler 910, the thermoelectric cooler 910 cools the charge coil 904, and on the opposite side of the thermoelectric cooler 910, the heatsink 906 dissipates excess heat from the thermoelectric cooler 910. Using the thermoelectric cooler 910 enables simultaneous charging and heat removal, which can more effectively cool the charge coil 904 compared to using the heatsink 906 alone.

### Example Device

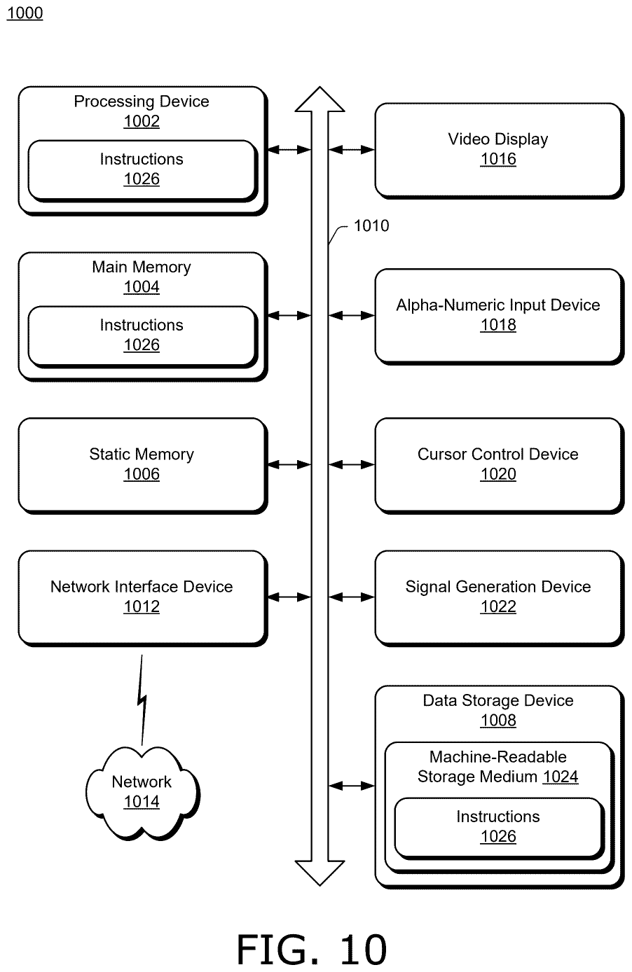

FIG. 10 illustrates a block diagram of an example computing device 1000 that can perform one or more of the operations described herein, in accordance with some implementations. The computing device 1000 can be connected to other computing devices in a local area network (LAN), an intranet, an extranet, and/or the Internet. The computing device 1000 can operate in the capacity of a server machine in a client-server network environment or in the capacity of a client in a peer-to-peer network environment. The computing device 1000 can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1000 is one or more of an ultrasound machine, an ultrasound scanner, an access point, a charging station, and a medical archiver.

The example computing device 1000 can include a processing device 1002 (a general-purpose processor, a programmable logic device (PLD), etc.), a main memory 1004 (synchronous dynamic random-access memory (DRAM), read-only memory (ROM), etc.), and a static memory 1006 (flash memory, a data storage device 1008, etc.), which can communicate with each other via a bus 1010. The processing device 1002 can be provided by one or more general-purpose processing devices such as a microprocessor, a central processing unit, or the like. In an illustrative example, the processing device 1002 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1002 can also comprise one or more special-purpose processing devices such as an ASIC, an FPGA, a digital signal processor (DSP), a network processor, or the like. The processing device 1002 can be configured to execute the operations described herein, in accordance with one or more aspects of the present disclosure, for performing the operations and steps discussed herein.

The computing device 1000 can further include a network interface device 1012, which can communicate with a network 1014. The computing device 1000 also can include a video display unit 1016 (a liquid crystal display (LCD), an organic light-emitting diode (OLED), a cathode ray tube (CRT), etc.), an alphanumeric input device 1018 (e.g., a keyboard), a cursor control device 1020 (e.g., a mouse), and an acoustic signal generation device 1022 (a speaker, a microphone, etc.). In one embodiment, the video display unit 1016, the alphanumeric input device 1018, and the cursor control device 1020 can be combined into a single component or device (e.g., an LCD touch screen).

The data storage device 1008 can include a computer-readable storage medium 1024 on which can be stored one or more sets of instructions 1026 (e.g., instructions for carrying out the operations described herein, in accordance with one or more aspects of the present disclosure). The instructions 1026 can also reside, completely or at least partially, within the main memory 1004 and/or within the processing device 1002 during execution thereof by the computing device 1000, where the main memory 1004 and the processing device 1002 also constitute computer-readable media. The instructions can further be transmitted or received over the network 1014 via the network interface device 1012.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1008 of the computing device 1000 as executable instructions or code. Although represented as software implementations, the described modules can be implemented as any form of a control application, a software application, a signal processing and control module, hardware, or firmware installed on the computing device 1000.

While the computer-readable storage medium 1024 is shown in an illustrative example to be a single medium, the term “computer-readable storage medium” should be taken to include a single medium or multiple media (e.g., a centralized or distributed database and/or associated caches and servers) that store the one or more sets of instructions 1026. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example User Interface

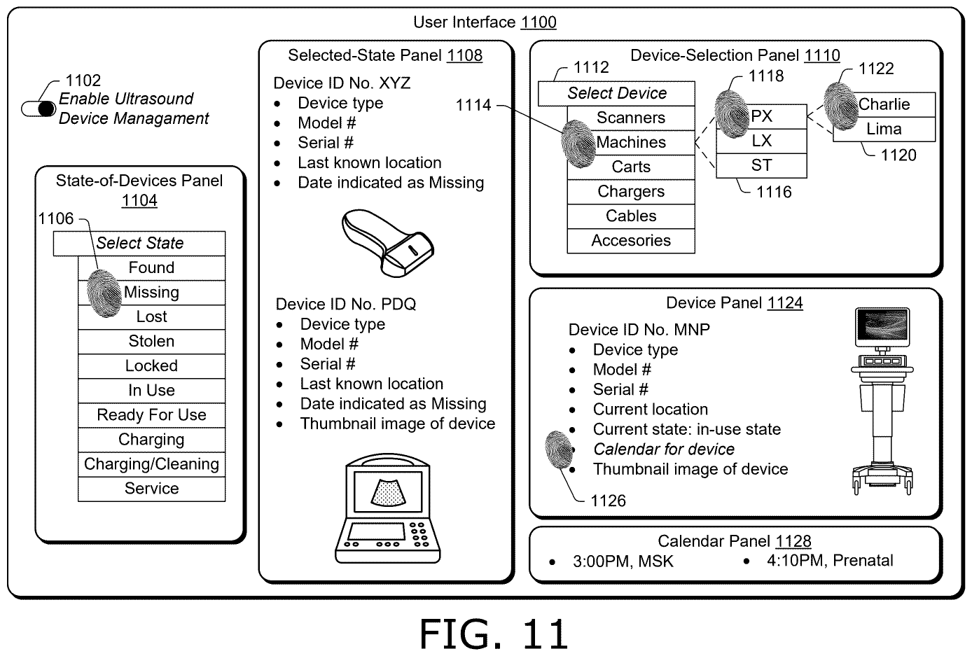

FIG. 11 illustrates an example user interface 1100 for managing ultrasound devices. The user interface 1100 can be displayed on any suitable computing device, such as the ultrasound machine 102, the computing device 510, the server system 812, and the like. The user interface 1100 includes a control option 1102 (e.g., a switch) to enable ultrasound device management in accordance with disclosed implementations. In response to the control option 1102 being enabled, the user interface 1100 can display a state-of-devices panel 1104.

The state-of-devices panel 1104 includes a drop-down menu with user-selectable options to select a state (e.g., a state of the state machine 300) in which an ultrasound device can be. In the example in FIG. 11, a user has selected a missing state, as indicated by a touch input 1106. Responsive to the selection indicated by the touch input 1106, the user interface 1100 displays a selected-state panel 1108. In this example, the selected-state panel 1108 corresponds to the selected missing state.

The selected-state panel 1108 displays ultrasound devices and information for these devices that are currently in the state of the state machine 300 that was selected in the state-of-devices panel 1104. In this example, the selected-state panel 1108 indicates two ultrasound devices that are in the missing state, including an ultrasound scanner with device-identification number XYZ and an ultrasound machine with device-identification number PDQ. The selected-state panel 1108 can display any suitable information for ultrasound devices in the selected state. Example information for the ultrasound devices in the missing state includes a device type (e.g., ultrasound scanner or ultrasound machine), a model number of the device, a serial number of the device (e.g., the manufacturer serial number, which can be different from the device-identification number PDQ, for example, a resource locator for a care facility), a last known location for the device, the date the device entered the missing state, etc. The selected-state panel 1108 can also display an image of the devices that are in the missing state.

Additionally or alternatively to displaying the state-of-devices panel 1104, responsive to the control option 1102 being enabled, the user interface 1100 can display a device-selection panel 1110. The device-selection panel 1110 can include any suitable option for a user to enter a device name to enable the system to display data regarding the current state of a device associated with the device name. For instance, the device-selection panel 1110 can include an option for a user to enter a device-identification number or serial number for a device, in case such data is known to the user. In the example illustrated in FIG. 11, the device-selection panel 1110 includes a first drop-down menu 1112 to select a device type, including ultrasound scanners, ultrasound machines, ultrasound carts, ultrasound chargers, ultrasound cables, and ultrasound accessories. Further, a user selects, via a touch input 1114, a device type of ultrasound machines. Responsive to the selection of the ultrasound machines type, the device-selection panel 1110 displays a drop-down menu 1116 that indicates selections for PX ultrasound machines, LX ultrasound machines, and ST ultrasound machines. The user then selects, via a touch input 1118, PX type ultrasound machines. In response to the selection of the PX type ultrasound machines, the device-selection panel 1110 displays a drop-down menu 1120 that indicates names of PX ultrasound machines in the care facility, including a PX ultrasound machine named Charlie and another PX ultrasound machine named Lima. In the illustrated example, the user selects, via a touch input 1122, the PX ultrasound machine named Charlie.

Responsive to the selection indicated via the touch input 1122 that selected the specific ultrasound device (e.g., the PX ultrasound machine named Charlie), the user interface 1100 displays a device panel 1124. The device panel 1124 can display any suitable data related to the state of the selected device, namely the PX ultrasound machine named Charlie. For instance, the device panel 1124 can display a device-identification number for the PX ultrasound machine named Charlie, the device type, the model number, the serial number, the current location of the device, the current state of the device in the state machine 300 (e.g., the in-use state 312 is indicated), a calendar (or schedule) for the device, and a thumbnail image of the device.

In the example in FIG. 11, the calendar for the PX ultrasound machine named Charlie displayed in the device panel 1124 is selectable (as evidenced by italics). In the illustrated example, the user selects, via a touch input 1126, the calendar for the PX ultrasound machine named Charlie. Responsive to the selection of the calendar for the device, the user interface 1100 displays a calendar panel 1128. The calendar panel 1128 can display any suitable data regarding the calendar or scheduling for the device. In the example in FIG. 11, the calendar panel 1128 indicates that the PX ultrasound machine named Charlie is scheduled for an MSK examination at 3:00 PM and a prenatal examination at 4:10 PM.

### Example Methods

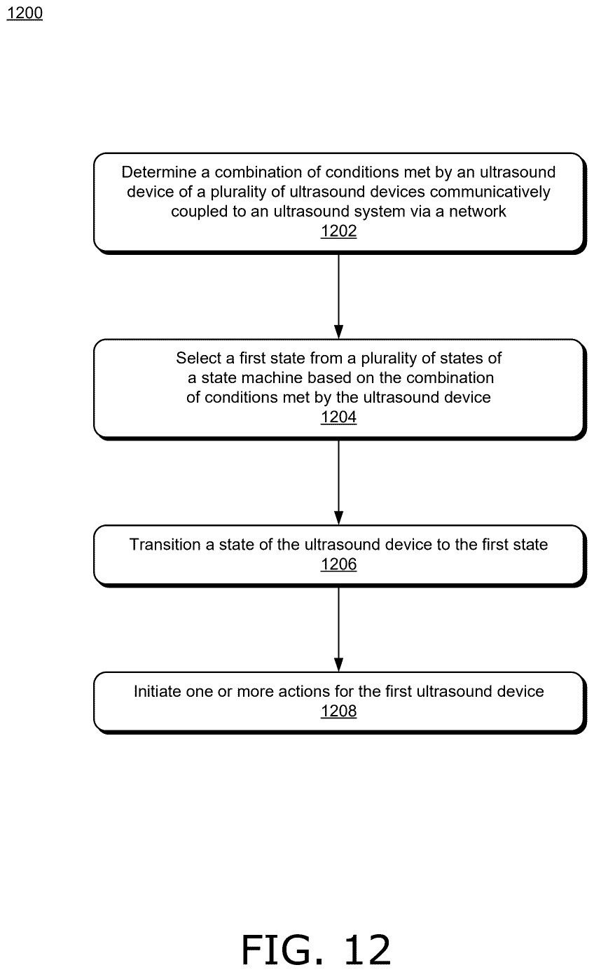

FIGS. 12-14 depict methods 1200, 1300, and 1400, respectively, for managing ultrasound devices. The methods 1200, 1300, and 1400 are shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example system 100 of FIG. 1 or to entities or processes as detailed in FIGS. 2-11, reference to which is made for example only. The techniques are not limited to performance by one entity or multiple entities operating on one device.

FIG. 12 depicts a method 1200 for managing ultrasound devices, in accordance with one or more implementations. The method 1200 can be performed by the ultrasound system 100. At 1202, a combination of conditions met by an ultrasound device of a plurality of ultrasound devices communicatively coupled to an ultrasound system via a network is determined. The combination of conditions can be determined by a state machine of the ultrasound system. Examples of the ultrasound system include the ultrasound system 100 in FIG. 1 and the ultrasound system 806 of FIG. 8. An example of the state machine is the state machine 300 of FIG. 3. The ultrasound system can monitor multiple ultrasound devices using the state machine. As disclosed herein, the combination of conditions can include a variety of conditions that indicate a particular state of the state machine for the corresponding ultrasound device.

At 1204, a first state is selected from a plurality of states of the state machine based on the combination of conditions met by the ultrasound device. In aspects, each state of the plurality of states corresponds to a different combination of conditions. Example states and corresponding conditions are described with respect to FIG. 3.

At 1206, a state of the ultrasound device is transitioned to the first state. For example, due to the combination of conditions met by the ultrasound device, the state machine determines and selects the first state to assign to the ultrasound device and then transitions (e.g., changes, assigns) the state of the ultrasound device to the first state from a previous state that is different from the first state, such that the first state becomes the current state of the ultrasound device.

At 1208, one or more actions are initiated for the ultrasound device. In aspects, the one or more actions are initiated responsive to transitioning the ultrasound device to the first state. The one or more actions correspond to the first state, such that the ultrasound system can initiate appropriate actions for the ultrasound device, such as specific actions to recover a lost ultrasound device or a stolen ultrasound device or specific actions to unlock an ultrasound device that was previously missing and locked and has now been located (e.g., found). Additional examples of such actions are described with respect to FIG. 3.

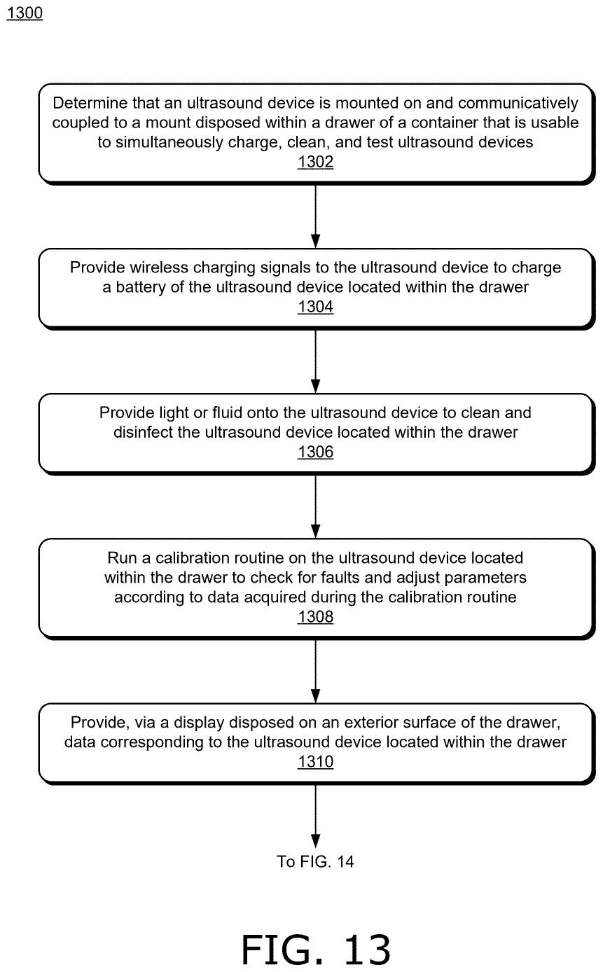

FIG. 13 depicts a method 1300 for managing ultrasound devices, in accordance with one or more implementations. The method 1300 can be performed by the ultrasound system 100. At 1302, an ultrasound device is determined to be mounted on and communicatively coupled to a mount disposed within a drawer of a container that is usable to simultaneously charge, clean, and test ultrasound devices. For example, the system 100 can receive an indication from the mount 708 that the ultrasound device (e.g., ultrasound scanner 710) is coupled to the mount 708.

At 1304, wireless charging signals are provided, via one or more charger transmitters disposed within the drawer, to the ultrasound device to charge a battery of the ultrasound device located within the drawer. For example, the mount 708 can include a charger array or a charger transmitter that provides charge to a battery of the ultrasound device. Alternatively or additionally, the charger transmitters 712-1 and 712-2 can be implemented in the drawer 704 to provide wireless charging signals to the ultrasound device (e.g., the ultrasound scanner 710).

At 1306, light from a cleaning source or fluid from a fluid source is provided onto the ultrasound device to clean and disinfect the ultrasound device located within the drawer. The cleaning fluid can be provided to rinse the ultrasound device, prior to and/or subsequent to applying disinfectant onto the ultrasound device. In some aspects, the cleaning fluid includes disinfectant fluid provided to disinfect the ultrasound device.

At 1308, a calibration routine is run on the ultrasound device located within the drawer to check for faults and adjust parameters according to data acquired during the calibration routine. The calibration routine includes a fault check on array elements of the ultrasound device. For example, the ultrasound system 100 removes fluid from the drawer that was provided via the fluid source and applies test signals to the ultrasound device. The test signals are used to determine an amount of capacitance across array elements of a transducer of the ultrasound device, which is then used to identify whether any of the array elements are faulty.

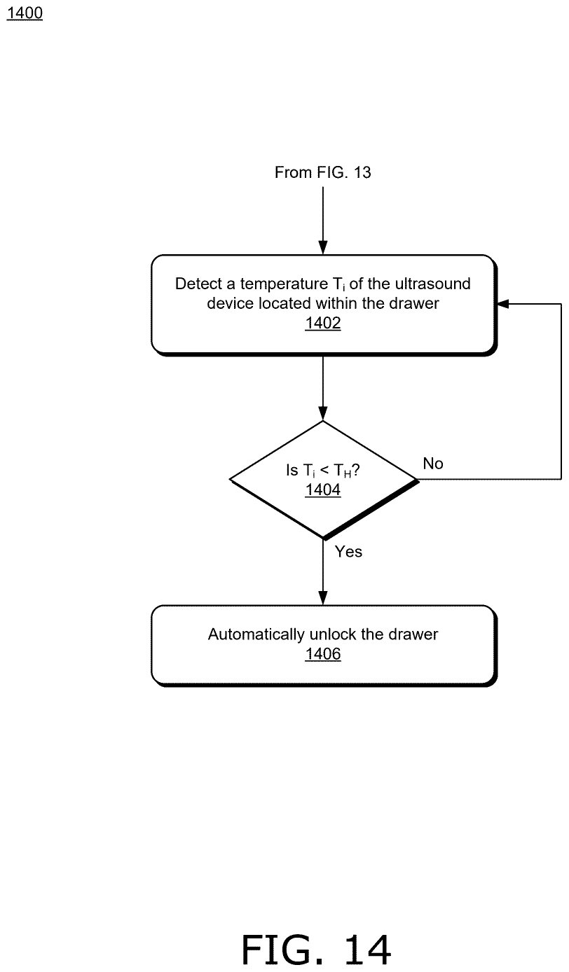

At 1310, data corresponding to the ultrasound device located within the drawer is provided via a display disposed on an exterior surface of the drawer, the data including a state of the ultrasound device in a state machine. The data displayed via the display (e.g., the display 724) can include a variety of information about the ultrasound device. The state of the ultrasound device in the state machine 300, for example, can be the charging/cleaning state if the charging, cleaning, and testing cycle is currently active. Alternatively, the state can be the ready-for-use state if the charging, cleaning, and testing cycle is complete. The display can also provide a thumbnail image of the ultrasound device located within the drawer to provide visual identification of the ultrasound device within the drawer, particularly for when the drawer is locked. In some implementations, the method 1300 continues to the method 1400 in FIG. 14.

FIG. 14 depicts a method for managing ultrasound devices, in accordance with one or more implementations. At 1402, a temperature Tᵢ of the ultrasound device located within the drawer is detected. The temperature Tᵢ of the ultrasound device can be detected in any suitable way, including using thermal sensors (e.g., thermistors) on the ultrasound device, an IR camera in the drawer, etc. The temperature Tᵢ of the ultrasound device can be detected and monitored at any time while the ultrasound device is located within the drawer, such as before, during, and/or after any of charging, cleaning, and/or testing of the ultrasound device.

At 1404, a determination is made as to whether the temperature Tᵢ of the ultrasound device is below a temperature threshold T_{H}. Such a determination can be made in any suitable way, examples of which including that the temperature Tᵢ can be compared to a numerical value representing the temperature threshold T_{H}, a specific resistance corresponding to the temperature threshold T_{H}, and a lookup table having the temperature threshold T_{H}.

If the temperature Tᵢ of the ultrasound device is above the temperature threshold T_{H} (“NO” at 1404), then the method 1400 loops back to 1402 to continue monitoring the temperature Tᵢ of the ultrasound device until the temperature cools to a safe temperature (e.g., below the temperature threshold T_{H}). For example, for safety and security, the drawer is locked during the charging, cleaning, and/or testing operations. Upon completion of the charging, cleaning, and testing operations, the temperature of the ultrasound device can be too high for a user to touch without being harmed. Accordingly, the system delays physical access to the ultrasound device (e.g., the drawer remains locked) until the temperature of the ultrasound device has cooled to a safe temperature.

If the temperature Tᵢ of the ultrasound device is at or below the temperature threshold T_{H} (“YES” at 1404), then at 1406, the drawer is automatically unlocked to enable access to the ultrasound device such that a user can remove the ultrasound device from the drawer.

While the present subject matter has been described in detail with respect to various specific example implementations thereof, each example is provided by way of explanation and not limitation of the disclosure. Those skilled in the art, upon attaining an understanding of the foregoing, can readily produce alterations to, variations of, and equivalents to such implementations. Accordingly, the subject disclosure does not preclude inclusion of such modifications, variations, and/or additions to the present subject matter as would be readily apparent to one of ordinary skill in the art. For instance, features illustrated or described as part of one implementation can be used with another implementation to yield a still further implementation. Thus, it is intended that the present disclosure cover such alterations, variations, and equivalents.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (a-a, a-a-a, a-a-b, a-a-c, a-b-b, a-c-c, b-b, b-b-b, b-b-c, c-c, and c-c-c or any other ordering of a, b, and c).

While various embodiments of the disclosure are described in the foregoing description and shown in the drawings, it is to be distinctly understood that this disclosure is not limited thereto but may be variously embodied to practice within the scope of the following claims. From the foregoing description, it will be apparent that various changes may be made without departing from the spirit and scope of the disclosure as defined by the following claims.

### CONCLUSION

Embodiments for managing ultrasound devices are disclosed. The techniques disclosed herein provide solutions for tracking the ultrasound devices and managing a state of each ultrasound device on a state machine. These techniques can enable a system to determine when a missing ultrasound device is lost versus stolen and further enable actions to be initiated based on the state of an ultrasound device to enable fast recovery of lost devices and reduce incentives for theft of devices. These techniques also include solutions for cleaning, disinfecting, and charging the ultrasound devices to increase the likelihood of an ultrasound device being ready for use, which enables better care to be provided to patients.

What is claimed is:

1. An ultrasound system comprising:
a state machine configured to manage a state of one or more ultrasound devices connected to a network, the state machine configured to:
determine a combination of conditions met by an ultrasound device of the one or more ultrasound devices;
determine a state of the ultrasound device based on the combination of conditions, different combinations of conditions each being associated with a different state of a plurality of states; and
initiate one or more actions based on the state of the ultrasound device.

2. The ultrasound system of claim 1, wherein:
the state of the ultrasound device is a current state that is different from a previous state of the ultrasound device prior to the combination of conditions being met; and
the one or more actions include changing the state of the ultrasound device from the previous state to the current state.

3. The ultrasound system of claim 1, wherein:
the plurality of states include a missing state and a found state;
a first condition for the missing state includes a location of the ultrasound device being unknown; and
a second condition for the found state includes the location of the ultrasound device being known.

4. The ultrasound system of claim 3, wherein:
the missing state includes multiple sub-states including a lost state, a stolen state, and a locked state;
the lost state is associated with a first combination of conditions including the location of the ultrasound device being unknown and a determination that the ultrasound device has not been stolen;
the stolen state is associated with a second combination of conditions including the location of the ultrasound device being unknown and a determination that the ultrasound device has been stolen; and
the locked state is associated with a third combination of conditions including the location of the ultrasound device being unknown and the ultrasound device being disabled to cause the ultrasound device to be rendered unsuitable for its intended purpose.

5. The ultrasound system of claim 3, wherein:
the found state includes sub-states including an in-use state, a ready-for-use state, and a service state;
the in-use state is associated with a first combination of conditions including the location of the ultrasound device being known and a determination that the ultrasound device is currently being used by an operator;
the ready-for-use state is associated with a second combination of conditions including the location of the ultrasound device being known and a determination that the ultrasound device is in a configuration in which the ultrasound device is usable by the operator; and
the service state is associated with a third combination of conditions including the ultrasound device being removed from availability for use and being sent for repairs or being repaired.

6. The ultrasound system of claim 5, wherein:
the plurality of states include a charging/using state and a charging/cleaning state;
the charging/using state is associated with a fourth combination of conditions including the location of the ultrasound device being known and the ultrasound device being currently in use and currently being charged; and
the charging/cleaning state is associated with a fifth combination of conditions including the location of the ultrasound device being known and the ultrasound device currently being at least one of cleaned, charged, or tested.

7. The ultrasound system of claim 1, further comprising:
the ultrasound device, the ultrasound device having a battery; and
a charger receiver connected to the ultrasound device and configured to charge the battery of the ultrasound device based on wireless charging signals received from a charger transmitter.

8. The ultrasound system of claim 7, further comprising:
an ultrasound cart having a stand head on which the ultrasound device is mountable, the stand head coupled to the charger transmitter, wherein the charger transmitter is configured to implement a non-contact charging system with the charger receiver to charge the battery of the ultrasound device when the ultrasound device is mounted on the stand head.

9. The ultrasound system of claim 7, wherein:
the charger transmitter is mounted on, in, or under a mounting surface; and
the charger transmitter is configured to implement a non-contact charging system with the charger receiver to charge the battery of the ultrasound device when the ultrasound device is within a threshold distance that is perpendicular to the mounting surface.

10. The ultrasound system of claim 9, further comprising:
a control system configured to:
receive position data of the ultrasound device from the charger transmitter when the ultrasound device is coupled to the charger transmitter;
provide charging control data to the charger transmitter for charging the ultrasound device based on the position data; and
receive charging status data of the ultrasound device from the charger transmitter.

11. The ultrasound system of claim 1, further comprising:
a display device; and
a user interface displayable via the display device, wherein the user interface includes:
a first panel having a plurality of user-selectable options to select a first state of the plurality of states; and
a second panel configured to, responsive to a user input selecting the first state in the first panel, display one or more ultrasound devices that are currently in the first state.

12. The ultrasound system of claim 1, further comprising:
a display device; and
a user interface displayable via the display device, wherein the user interface includes:
a device-selection panel having options for a user input to enter a device name; and
a device panel configured to, responsive to the user input entering the device name in the device-selection panel, display data corresponding to a device associated with the device name, the data including one or more of a device-identification number, a device type, a model number, a current location, a calendar for the device, an image of the device, and the state of the device.

13. The ultrasound system of claim 1, further comprising:
one or more processors configured to transmit instructions to the ultrasound device that cause the ultrasound device to activate a lightbar of the ultrasound device to indicate the state of the ultrasound device.

14. A system comprising:
a container having a drawer for storing one or more ultrasound devices, the drawer including:
a mount configured to couple to an ultrasound device;
a charger transmitter configured to provide wireless charging signals to the ultrasound device coupled to the mount;
one or more cleaning sources configured to emit a light to clean and disinfect the ultrasound device coupled to the mount; and
a fluid source configured to emit a fluid into the drawer to clean and disinfect the ultrasound device coupled to the mount.

15. The system of claim 14, further comprising:
a calibration tool holder; and
a calibration tool coupled to the calibration tool holder, the calibration tool configured to run a calibration routine on the ultrasound device coupled to the mount to check for faults and adjust parameters according to data acquired during the calibration routine.

16. The system of claim 14, further comprising:
a state machine configured to manage a state of the one or more ultrasound devices connected to a network, wherein the one or more ultrasound devices include the ultrasound device coupled to the mount within the drawer, the state machine configured to:
determine a combination of conditions met by the ultrasound device;
determine the state of the ultrasound device based on the combination of conditions, different combinations of conditions each being associated with a different state of a plurality of states; and
initiate one or more actions based on the state of the ultrasound device.

17. The system of claim 16, further comprising:
a display device disposed on an exterior surface of the drawer, the display device configured to indicate data related to the ultrasound device coupled to the mount within the drawer, the data including the state of the ultrasound device.

18. The system of claim 14, further comprising:
one or more thermal sensors configured to detect a temperature of the ultrasound device coupled to the mount within the drawer; and
a processor configured to:
cause the drawer to be locked during a maintenance cycle that includes at least one of charging, cleaning, or testing of the ultrasound device; and
cause the drawer to be automatically unlocked responsive to the maintenance cycle being completed and the temperature of the ultrasound device being below a temperature threshold.

19. A method for managing ultrasound devices, the method comprising:
determining, by a state machine of an ultrasound system, a combination of conditions met by an ultrasound device communicatively coupled to the ultrasound system via a network;
selecting a first state from a plurality of states of the state machine based on the combination of conditions met by the ultrasound device, each state of the plurality of states corresponding to a different combination of conditions;
transitioning a state of the ultrasound device to the first state; and
responsive to transitioning the ultrasound device to the first state, initiating one or more actions for the ultrasound device.

20. The method of claim 19, wherein the one or more actions include:
one or more first actions to recover the ultrasound device if the first state is a lost state;
one or more second actions to disable the ultrasound device and remove at least some economic value of the ultrasound device if the first state is a stolen state;
one or more third actions to unlock the ultrasound device if the first state is a found state and the ultrasound device was previously in a locked state;
one or more fourth actions to notify a user that the ultrasound device is ready for use if the first state is a ready-for-use state; or
one or more fifth actions to prepare the ultrasound device for repair if the first state is a service state.
